(19) United States
(12) Patent Application Publication
(10) Pub. No.: US 2023/0346842 A1
(43) Pub. Date: Nov. 2, 2023

(54) Methods and Compositions For Reducing Joint Inflammation Using Mesenchymal Stem Cells

(71) Applicant: PrimeGen Biotech, LLC, Santa Ana, CA (US)

(72) Inventors: Fariborz Izadyar, Santa Ana, CA (US); Chad Maki, Santa Ana, CA (US); Thomas Yuen, Santa Ana, CA (US); Raymond Tong, Santa Ana, CA (US)

(21) Appl. No.: 17/918,699

(22) PCT Filed: May 7, 2021
(86) PCT No.: PCT/US2021/031288

§ 371 Date: Oct. 13, 2022

Related U.S. Application Data
(60) Provisional application No. 63/021,268, filed on May 7, 2020.

(51) Int. Cl. A61K 35/28; C12N 5/0775; A61P 13/12; A61P 29/00; A61P 37/00; A61P 19/02

(52) U.S. Cl. CPC A61K 35/28; C12N 5/0667; A61P 13/12; A61P 29/00; A61P 37/00; A61P 19/02

(57) ABSTRACT

Provided are methods of treatment comprising administering to a subject in need thereof a therapeutically effective amount of stem cells to the affected tissue or organ.

### TECHNICAL FIELD

This disclosure relates to methods and compositions for treating tissues using stem cells. More specifically described herein are treatment modalities employing mesenchymal stem cells (MSC) in the treatment of mammals, as well as MSC purification and formulation methods including the “activation” or “preconditioning” of stem cells.

### BACKGROUND

Stem cells are specialized cells, capable of renewing themselves through cell division as well as differentiating into multi-lineage cells. These cells can be categorized as embryonic stem cells (ESC), induced pluripotent stem cells (iPSC), and adult stem cells. Mesenchymal stem cells (MSC) are adult stem cells which can be isolated from human and animal sources. Human MSC (hMSC) are non-haematopoietic, multipotent stem cells with the capacity to differentiate into mesodermal lineage such as osteocytes, adipocytes and chondrocytes as well ectodermal (neurocytes) and endodermal lineages (hepatocytes). MSC express cell surface markers including cluster of differentiation (CD)29, CD44, CD73, CD90, CD105, and lack the expression of CD14, CD34, CD45, and HLA (human leucocyte antigen)-DR. hMSC have been isolated from various tissues, including adipose tissue, amniotic fluid, endometrium, dental tissues, umbilical cord, and Wharton's jelly. hMSC have been cultured long-term in specific media without any severe abnormalities.

Furthermore, MSC display immunomodulatory features, and can secrete cytokines and immune-receptors which regulate the microenvironment in the host tissue. Multilineage potential, immunomodulation and secretion of anti-inflammatory molecules makes MSC an effective tool in the treatment of chronic diseases.

For example, osteoarthritis (OA) or degenerative joint disease is the most common form of arthritis in dogs, affecting a quarter of the population. OA can occur as a primary disease in aging patients, but can also occur as a secondary disease in younger dogs. Primary OA is a result of normal or abnormal forces on an otherwise normal joint. This disease generally takes years of wear and tear before clinical symptoms manifest, but in young patients that have predisposing conditions such as hip dysplasia (HD) or elbow dysplasia (ED), symptoms may manifest as early as 1-2 years old. Secondary OA occurs as a result of an insult to the diseased joint, such as joint trauma or infection, followed by normal or abnormal forces on an abnormal joint. OA involves cartilage degeneration, fibrillation and loss, inflammation and hyperplasia of the synovial membrane, abnormal proliferation of bone (osteophyte production) and eventually exposure of subchondral bone (see FIG. 23).

OA is a debilitating disease, as it is progressive, degenerative, debilitating and painful and inevitably leads to joint failure. Non-Steroidal Anti-Inflammatory Drugs (NSAIDs) (such as grapiprant) and corticosteroids are often the first palliative medical treatment modality that is implemented for treatment of patients with OA. These therapies offer a temporary relief, but long-term utilization of them may have serious side effects. Other medical interventions include intra-articular administration of Hyaluronic Acid (HA) that acts as lubricant, and Interleukin 1 receptor antagonist (IRAP) that reduces inflammation.

None of these treatment modalities cure OA, and they require repeated administration. The only product on the market that asserts to be a disease-modifying drug is ADEQUAN® (polysulfated glycosaminoglycan (PSGAG)), which claims to restore joint lubrication, relieve inflammation and renew the building blocks of healthy cartilage. ADEQUAN® still requires repeat and continual administration with mixed results. Other supplements for OA include omega-3 fatty acid diets, chondroitin/glucosamine, and elk velvet and green-lipped muscle preparations. While these supplements may be helpful, they are not well-proven and do not produce a drastic effect.

### SUMMARY

The present disclosure is based, at least in part, on the non-limiting theory that MSC can be used to treat various conditions, for example conditions afflicting mammals, for example animals.

Disclosed embodiments comprise compositions for treating a patient, for example a mammal, suffering from a disease or diseased state, said compositions comprising MSC derived from progenitor cells isolated from, for example, adipose tissue, placental tissue, bone marrow, dental tissue, testicle tissue, uterine tissue, umbilical cord tissue, or skin tissue, that are allogeneic or autologous to a target patient; and a saline solution, wherein the composition can prevent, reduce, or eliminate the symptoms of one or more diseases or diseased states in a target patient, wherein the diseases or diseased states include, for example, immune system disorders, inflammatory diseases, and chronic diseases.

Embodiments comprise treatment of degenerative bone disease, osteoarthritis, rheumatoid arthritis, polyarthritis, systemic lupus erythematosus, inflammatory bowel disease, atopy, hepatitis, chronic steroid responsive meningitis-arteritis, beagle pain syndrome, degenerative myelopathy, chronic renal failure disease, dilated and mitral cardiomyopathy, keratoconjunctivitis sicca, immune mediated non-erosive arthritis, immune mediated hemolytic anemia, immune mediated thrombocytopenia, Evans syndrome, intervertebral disc disease, muscle fibrosis secondary to disease or trauma, refractory corneal ulcer, diabetes mellitus, spinal trauma, eosinophilic granuloma complex, hypertrophic cardiomyopathy, cholangitis, exercise induced pulmonary hemorrhage, rhabdomyolysis, corneal ulcer, eczema, multiple sclerosis, muscular dystrophy, spinal injury, hepatitis, myocardial infarction, heart disease, congestive heart failure, muscle fibrosis secondary to disease or trauma, and combinations thereof.

Disclosed embodiments comprise treatment of imflammatory disorders.

Disclosed embodiments comprise treatment of immune-related disorders.

Disclosed embodiments comprise treatment of musculoskeletal disorders.

Disclosed embodiments comprise methods of preventing or limiting an immune response.

Disclosed embodiments comprise isolation of stem cells, for example mesenchymal stem cells (MSC) from various tissues. Disclosed embodiments comprise methods of producing MSC formulations. Disclosed embodiments comprise methods of banking MSC. Disclosed embodiments comprise methods of propagating MSC.

Disclosed embodiments comprise therapeutic use of “activated” MSC. For example, embodiments comprise purifying MSC with different abilities to maximize their therapeutic benefit for specific use, for example using cell-sorting procedures such as Magnetic-Activated Cell Sorting (MACS) or Fluorescence-Activated Cell Sorting (FACS).

Further embodiments comprise activating MSC with specific stimulatory agents including, for example, cytokines, reactive proteins, chemicals, small molecules, and combinations thereof. These stimulatory agents can enhance or suppress MSC function; for example, immunosuppression by MSC is induced by proinflammatory cytokines. In further embodiments, MSC can be activated with energy, for example low-level laser energy or pulsed electromagnetic field energy.

### BRIEF DESCRIPTION OF THE DRAWINGS

FIG. 1 depicts the number of MSC isolated per gram of canine placenta.

FIG. 2 depicts the total number of MSC isolated per canine placenta.

FIG. 3 depicts MSC viability after isolation.

FIG. 4 depicts MSC proliferation pattern.

FIG. 5 depicts MSC doubling pattern.

FIG. 6 depicts a karyotype analysis of canine placental MSC.

FIG. 7 depicts a cell proliferation pattern of canine placental MSC.

FIG. 8 depicts an average cell doubling pattern of canine placental MSC.

FIG. 9 depicts an average cell proliferation pattern of canine placental MSC.

FIG. 10 depicts canine MSC cell surface markers.

FIG. 11 depicts canine MSC cell surface markers.

FIG. 12 depicts canine MSC cell surface markers.

FIG. 13 depicts differentiation of canine placental MSC to adipocytes.

FIG. 14 depicts differentiation of canine placental MSC to chondroocytes.

FIG. 15 depicts differentiation of canine placental MSC to osteocytes.

FIG. 16 depicts sterility testing of canine placental MSC.

FIG. 17 depicts MRL assay results of canine placental MSC.

FIG. 18 depicts secretion of IDO by canine placental MSC.

FIG. 19 depicts secretion of PGE2 by canine placental MSC.

FIG. 20 depicts canine placental MSC expansion strategies.

FIG. 21 depicts canine placental MSC expansion strategies.

FIG. 22 depicts canine placental MSC scalability.

FIG. 23 depicts pathologic changes in a joint as a result of osteoarthritis.

FIG. 24 depicts canine lameness improvement during the first 30 days.

FIG. 25 depicts canine lameness improvement after 90 days.

FIG. 26 depicts canine lameness improvement and MSC dose.

FIG. 27 depicts canine lameness improvement due to MSC administration.

FIG. 28 depicts serum level of IRAP in canine patients with improved lameness score.

FIG. 29 depicts serum level of IL-10 in canine patients with improved lameness score.

FIG. 30 depicts correlation between serum IL-10 and MSC dose.

FIG. 31 depicts changes in serum level of IL-10 in response to various doses of MSC.

FIG. 32 depicts Canine Brief Pain Inventory (CBPI) Pain Severity and Pain Interference Reduction scores of canines in different treatment groups.

FIG. 33 depicts CBPI Pain Severity and Pain Interference Reduction scores of canines in the placebo group as compared to MSC groups.

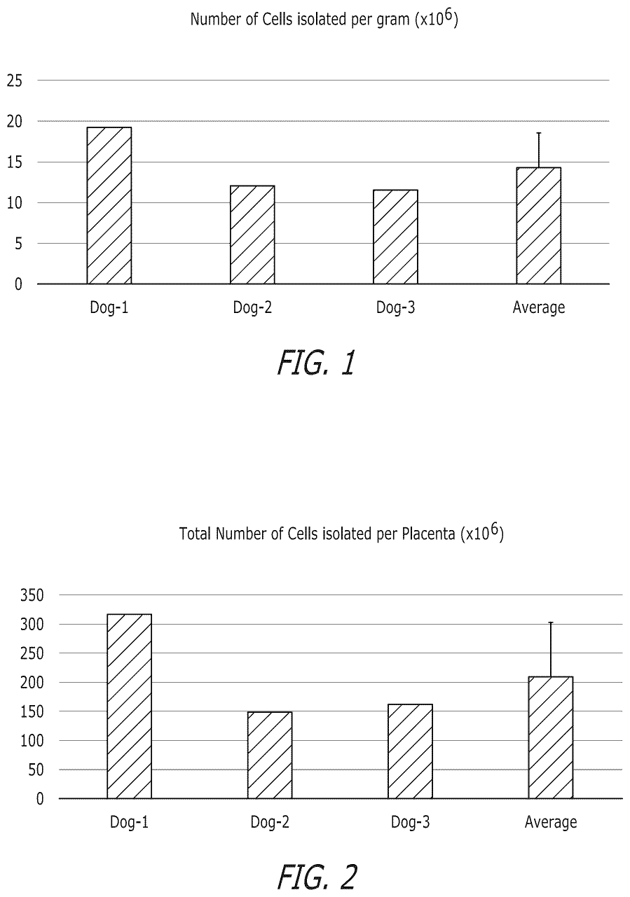

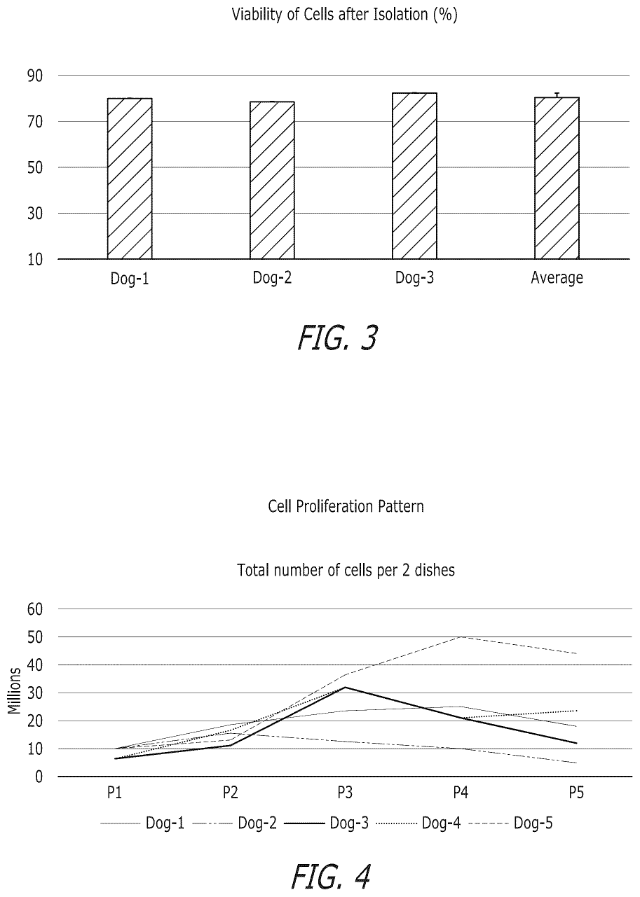

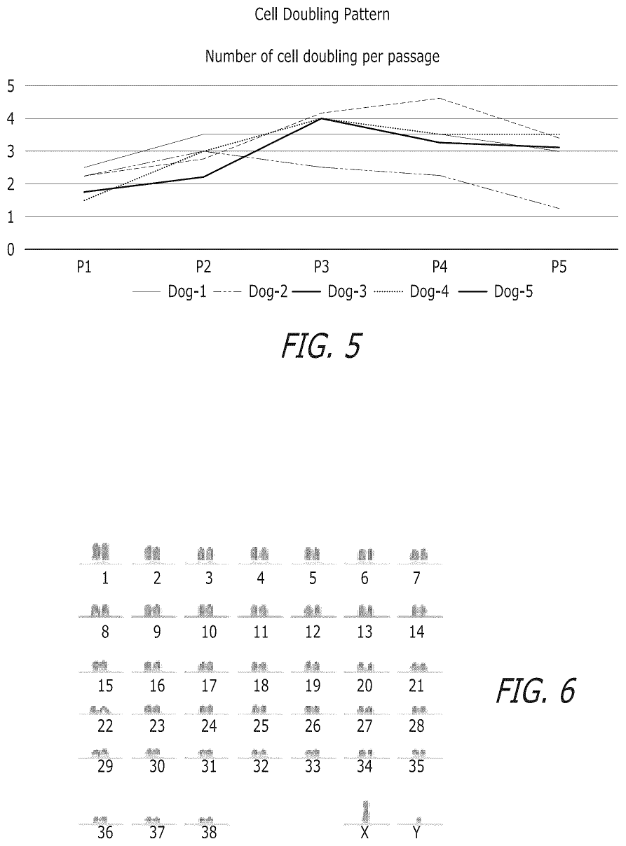

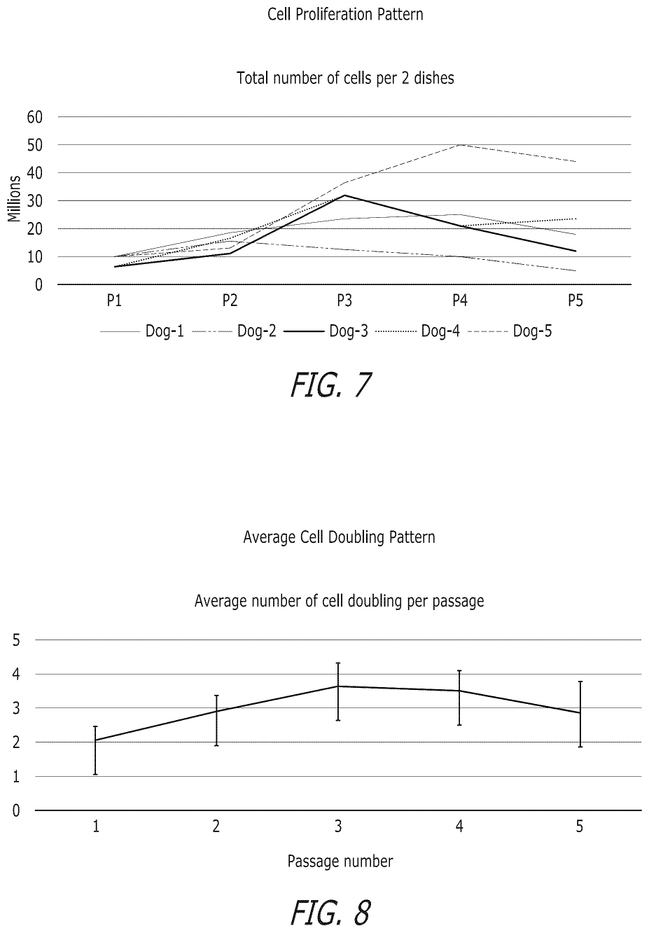

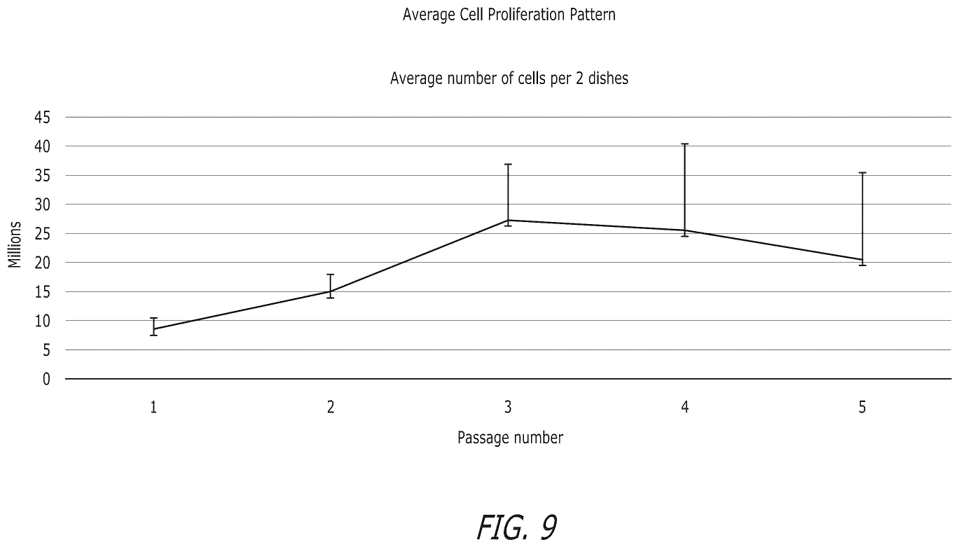

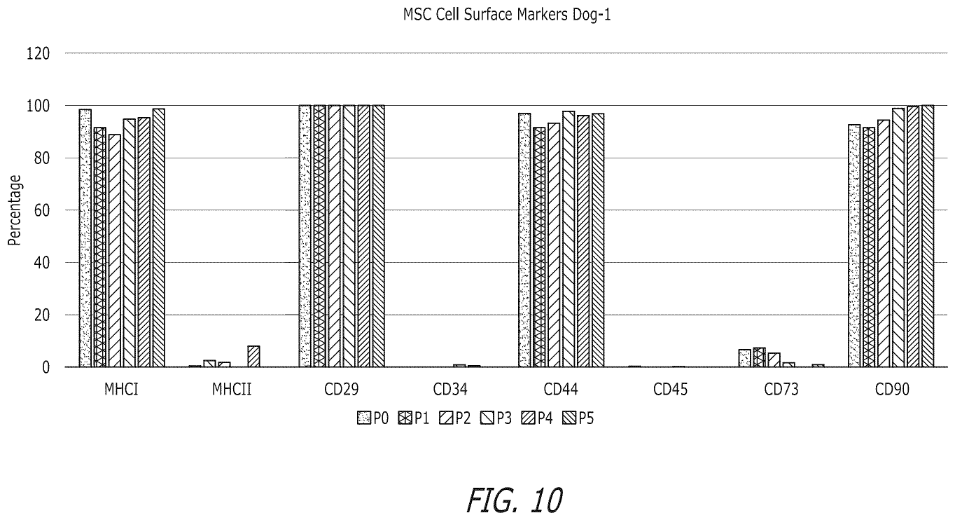

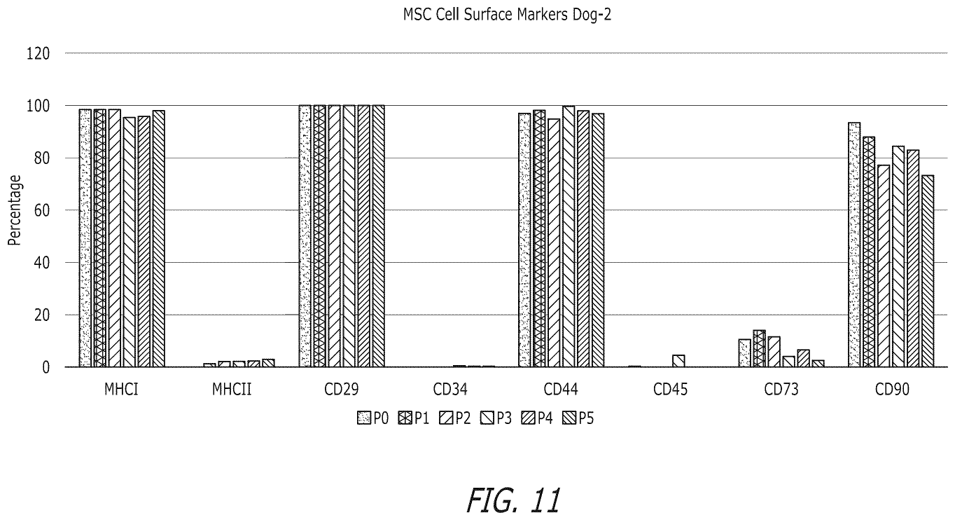

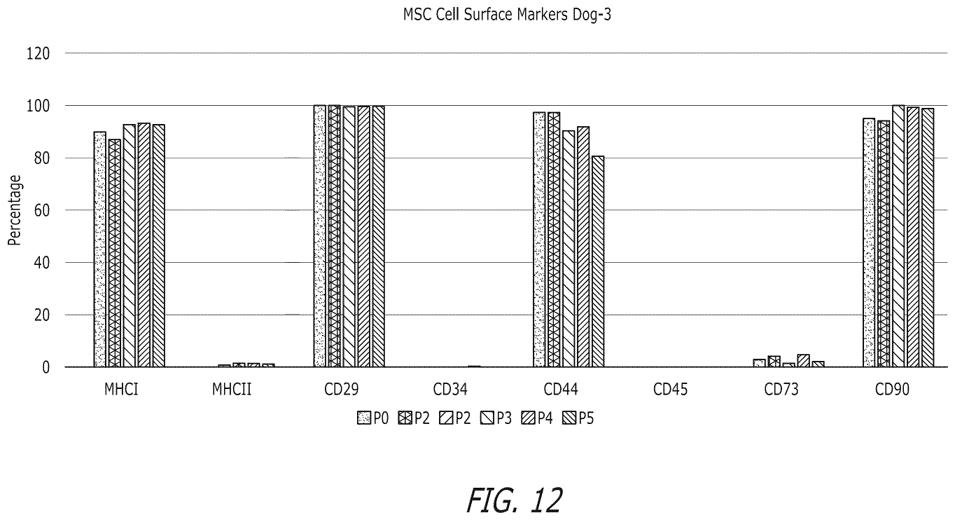

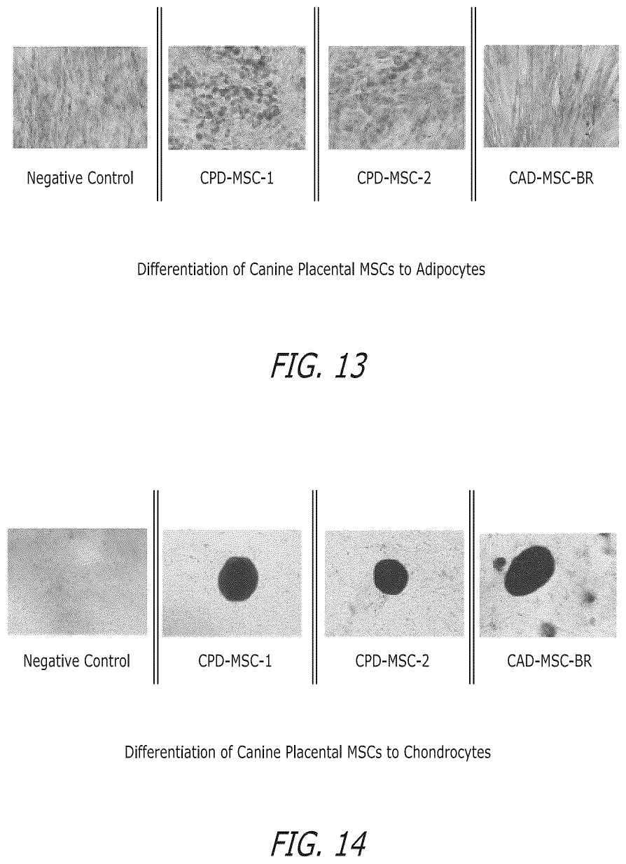

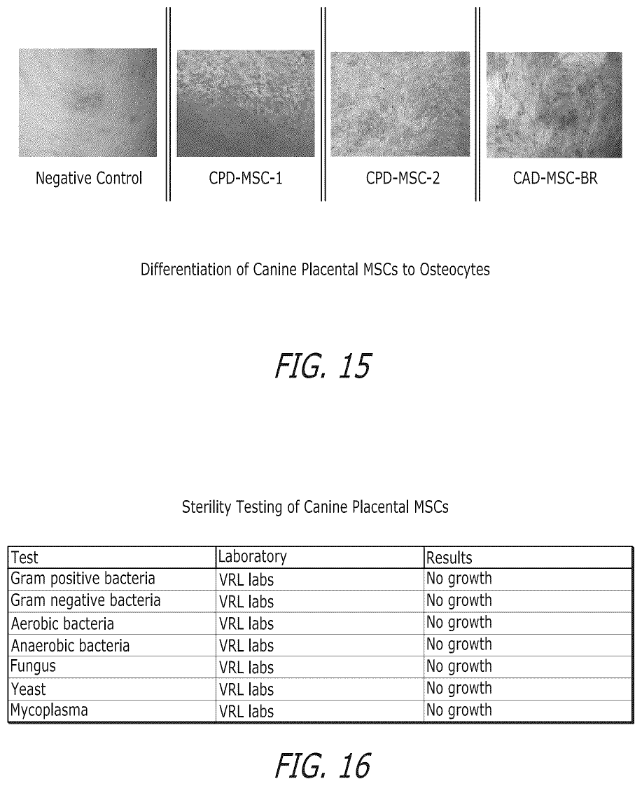

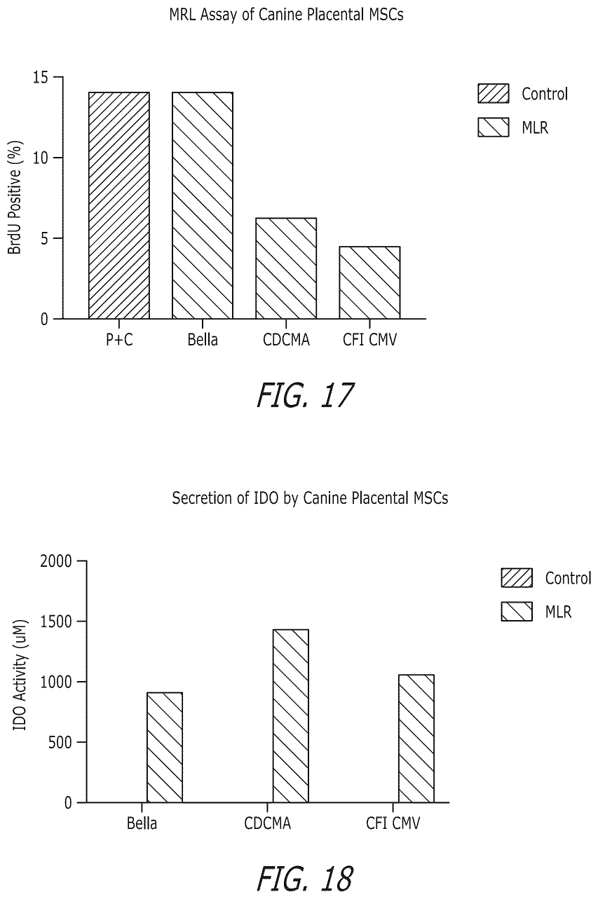

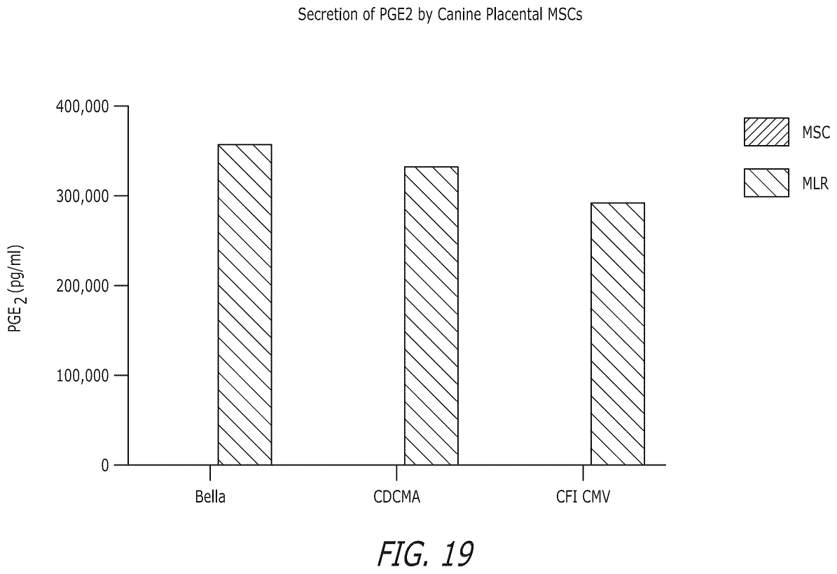

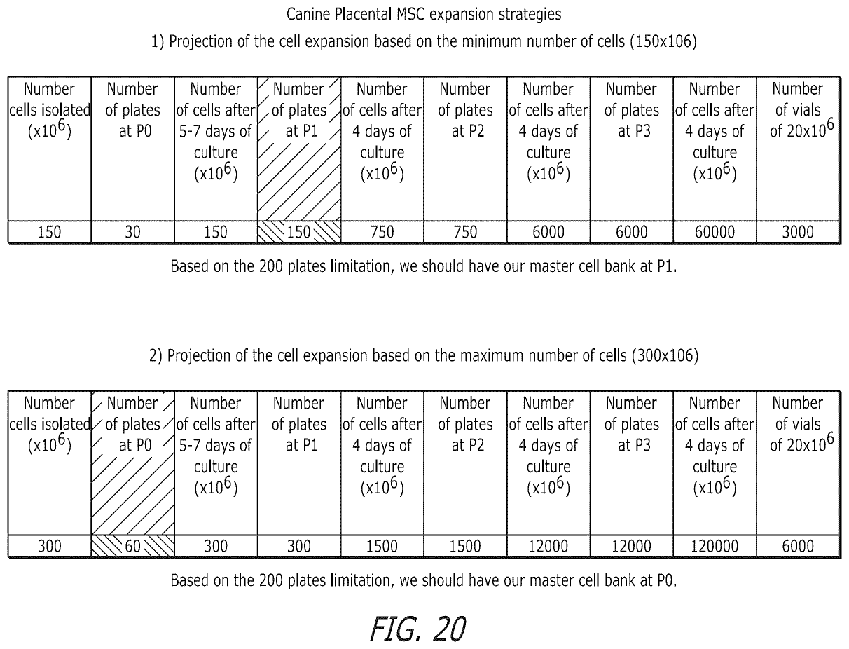

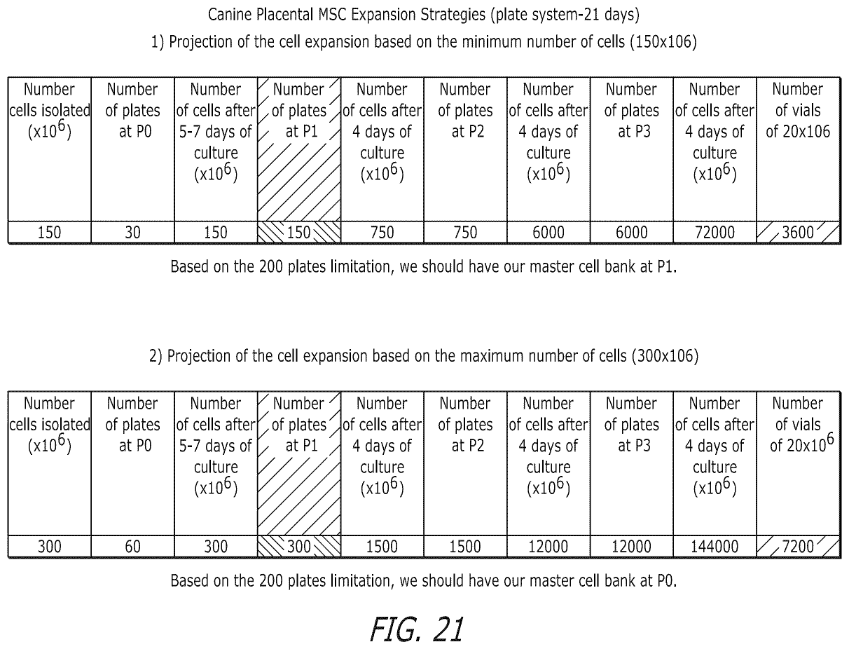

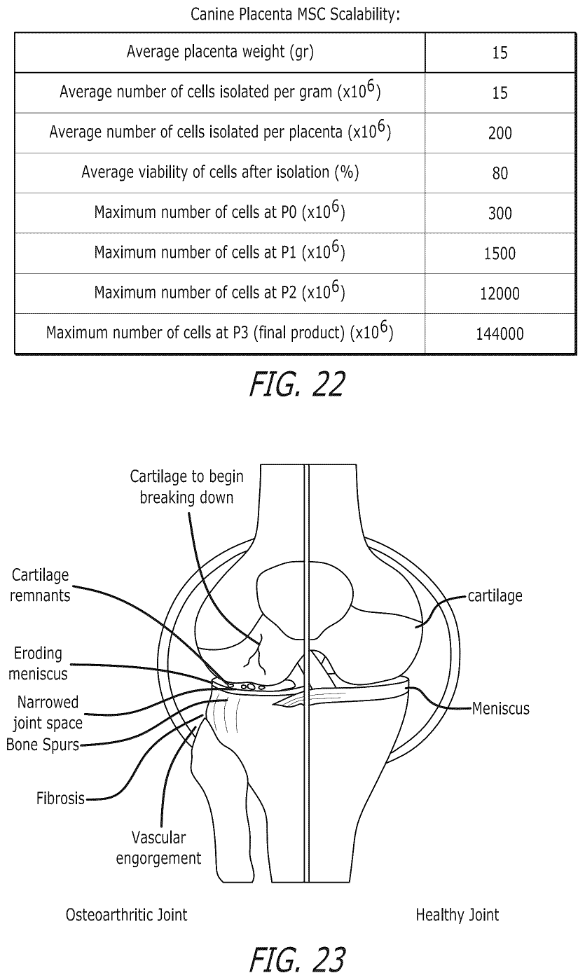

### DETAILED DESCRIPTION

The present disclosure is based, at least in part, on benefits of treating patients using MSC, for example placental-derived or adipose-derived MSC. Disclosed methods can include ameliorating or lessening pain or other disease or condition symptoms, for example lessening at least one symptom of an immune response or immune-related disorder. Subjects suitable for the disclosed methods can include, for example, mammals, such as animals, for example companion animals. Methods disclosed herein can comprise administration of other bioactive agents, for example an immunosuppressive agent.

Disclosed herein are methods of isolating and purifying MSC, for example placental MSC, adipose-derived MSC, and the like. Further embodiments comprise methods of propagating and banking MSC, for example placental-derived or adipose-derived MSC. Embodiments comprise purifying MSC based upon the cells' different abilities to maximize their therapeutic benefit for specific use, for example using cell-sorting procedures such as Magnetic-Activated Cell Sorting (MACS) or Fluorescence-Activated Cell Sorting (FACS).

Additional embodiments comprise activating MSC to modulate their therapeutic benefit, for example to increase their ability to suppress or enhance an immune response.

The articles “a” and “an” are used herein to refer to one or to more than one (i.e., to at least one) of the grammatical object of the article. By way of example, “an element” means one element or more than one element.

The term “activate” (or “pre-condition”) as used herein in the context of MSC refers to the use of stimulatory agents including, for example, cytokines, reactive proteins, chemicals, small molecules, and combinations thereof to enhance MSC function.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The term “treatment” or “treating” refers to any therapeutic intervention method in a mammal, for example a human or companion animal, including: (i) prevention, that is, causing the clinical symptoms not to develop, e.g., preventing infection or inflammation from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection.

The terms “reducing”, “suppressing” and “inhibiting” have their commonly understood meaning of lessening or decreasing.

The terms “effective,” “effective amount,” and “therapeutically effective amount” refer to that amount of MSC or a pharmaceutical composition thereof that produces a beneficial result after administration.

The phrases “parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “pharmaceutical composition” refers to a formulation containing the therapeutically active agents described herein in a form suitable for administration to a subject. In embodiments, the pharmaceutical composition is in bulk or in unit dosage form. The quantity of active ingredient (e.g., MSC) in a unit dose of composition is an effective amount and can be varied according to the particular treatment involved. One skilled in the art will appreciate that it is sometimes necessary to make routine variations to the dosage depending on the age and condition of the patient. The dosage will also depend on the route of administration. In preferred embodiments, the active ingredients are mixed under sterile conditions with a pharmaceutically acceptable carrier, and with any preservatives, buffers, or propellants that are required.

The terms “pharmaceutically acceptable” or “therapeutically acceptable” refers to a substance which does not interfere with the effectiveness or the biological activity of the active ingredients and which is not toxic to the host.

The phrase “pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

A “patient,” “subject,” or “host” to be treated by the subject method can mean, for example, a human or non-human animal, such as a mammal, a fish, a bird, a reptile, or an amphibian.

The term “in vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

### Isolation of MSC

Disclosed embodiments can comprise methods of harvesting and isolating MSC.

In disclosed embodiments, MSC can be harvested and isolated from a variety of tissues, including, but not limited to, placenta, skeletal muscle, adipose tissue, umbilical cord, synovium, the circulatory system (e.g., blood), dental pulp, amniotic fluid, fetal blood, lung, liver, gonadal tissue, and bone marrow. In embodiments, such methods can comprise aseptically collecting tissue from eligible mammalian donors.

For example, in embodiments utilizing placental MSC, tissue can be collected from full-term fetuses or during the third trimester of pregnancy. In embodiments, placenta is collected from specific pathogen-free donors, or from healthy donors with known health and travel history, free from adventitious agents. Multiple parts of placenta can be used for derivation of MSC, including, for example, endotheliochorial membrane, chorioallantoic membrane, amniotic membrane, umbilical cord, and Warthon's Jelly.

In embodiments comprising isolation of placental MSC, the following steps can be performed, for example in a certified clean room under cGMP conditions. Placenta tissue is washed extensively in rinsing buffer. Placenta tissue is then cut into small pieces (1-5 grams). Decidual giant cells are removed by one or a combination of steps, for example mechanical scraping of Decidual surface by sterile scoop. Placenta tissue is incubated with a protease, for example a serine protease, for example trypsin, for 30-90 minutes at 37° C. and 5% CO₂. Filtration using, for example, nylon mesh, for example 20, 25, and 30 micron nylon mesh, is then performed. Gradient separation of the cells is then performed, for example using BSA, Percoll, or Ficoll. Differential adhesion is used to allow the quick-attaching cells to be separated from the non-attached cells that are floating in the media. Placenta tissue is minced for, for example, 90 seconds or 150 cutting cycles.

In disclosed embodiments, placenta tissue is then subjected to digestion, for example enzymatic digestion, at 37° C. using an enzyme such as collagenase, for 60-180 min while shaking at the rate of, for example, 100 to 140 cycles per minute. Collagenase concentration for dog- and cat-derived placental tissue is typically 1 mg/ml, and for horse is typically 2.5 mg/ml. The cells are then passed through a sequence of cell strainers (for example, 100 micron, 40 micron, etc.) and then through a nylon mesh of, for example, 20, 25 or 30 microns. In some cases, cells will be passed through a gradient.

Red Blood Cells (RBC) are removed by RBC lysis buffer (3 min at 4° C.). RBC lysis is neutralized by adding PBS, for example 15-20 times PBS. Cells are then centrifuged at, for example, 400 g for 10 min. Cells are then cultured at a density of, for example, 200-300×10³ per cm² in a culture flask for a culture period of, for example, 5-7 days. In some cases, to remove the remaining giant cells from the mixture, differential adhesion will be implied, and cells will be allowed to attach for a few hours, and then the floating cells will be separated from the attached cells. After the culture period, placenta MSC can be harvested from the flask as P0 (passage zero).

In disclosed embodiments, MSC can be isolated from canine placenta in an amount of 1×10⁶ MSC per gram placenta, 2×10⁶ MSC per gram placenta, 3×10⁶ MSC per gram placenta, 4×10⁶ MSC per gram placenta, 5×10⁶ MSC per gram placenta, 6×10⁶ MSC per gram placenta, 7×10⁶ MSC per gram placenta, 8×10⁶ MSC per gram placenta, 9×10⁶ MSC per gram placenta, 1×10⁷ MSC per gram placenta, 2×10⁷ MSC per gram placenta, 3×10⁷ MSC per gram placenta, 4×10⁶ MSC per gram placenta, or the like.

In embodiments, MSC can demonstrate a viability after isolation of greater than 50%, greater than 60%, greater than 70%, greater than 80%, greater than 90%, or the like. In embodiments, MSC can demonstrate a viability after isolation of no less than 50%, no less than 60%, no less than 70%, no less than 80%, no less than 90%, or the like. In embodiments, MSC can demonstrate a viability after isolation of between 50% and 60%, between 60% and 70%, between 70% and 80%, between 80% and 90%, or between 90% and 100%.

MSC can be identified using the minimal criteria established by the Mesenchymal and Tissue Stem Cell Committee of the International Society for Cellular Therapy. These criteria include: first, MSC must be plastic-adherent when maintained in standard culture conditions; second, MSC must express CD105, CD73, and CD90, and lack expression of CD45, CD34, CD14 or CD11b, CD79α or CD19, and HLA-DR surface molecules; and third, MSC must be able to differentiate to osteoblasts, adipocytes and chondroblasts in vitro.

In embodiments, 100% of the MSC cells express CD29. In embodiments, expression of CD90 increases as the cells are passaged. In embodiments, expression of CD73 is below 20%.

In embodiments, MSC can be isolated based on their ability to produce therapeutic molecules, for example cytokines. For example, Magnetic-Activated Cell Sorting (MACS) can be used to purify MSC based on the cells' ability to produce a particular cytokine. Disclosed embodiments can also comprise the use of flow cytometry to purify MSC based on the cells' ability to produce a particular cytokine. Disclosed embodiments can also comprise the use of chromatography, for example affinity chromatography, to purify MSC based on the cells' ability to produce a particular cytokine.

### Expansion of MSC

In embodiments, cell expansion for cells originating from any of the above-mentioned tissues above takes place in clean room facilities purpose built for cell therapy manufacture and meeting GMP clean room classification. For example, in a sterile class II biologic safety cabinet located in a class 10,000 clean production suite, cells can be thawed under controlled conditions and washed in a 15 mL conical tube with 10 ML of complete DMEM-low glucose media (cDMEM) (GibcoBRL, Grand Island, N.Y.) supplemented with 10% Fetal Bovine Serum (Hyclone) specified to have endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments.

In embodiments, cells are subsequently placed in a T-225 flask containing 45 mL of cDMEM and cultured for 24 hours at 37° C. at 5% CO₂ in a fully-humidified atmosphere. This allows the MSC to adhere. Non-adherent cells are washed off using cDMEM by gentle rinsing of the flask. Adherent cells are subsequently detached by washing the cells with PBS and addition of 0.05% trypsin containing EDTA (Gibco, Grand Island, N.Y., USA) for 2 minutes at 37° C. at 5% CO₂ in a fully-humidified atmosphere. In embodiments. cells are centrifuged, washed and plated in T-225 flask in 45 mL of cDMEM.

In disclosed embodiments, this produces approximately 6 million cells per initiating T-225 flask. The cells of the first flask are then split into 4 flasks. Cells are grown for 4 days after which approximately 6 million cells per flask are present (24 million cells total). In embodiments, this method is repeated but cells are typically not expanded beyond 10 passages, and are then banked in 6 million cell aliquots in sealed vials for delivery.

In further embodiments, cells are grown in media and the cells, along with the media, are recovered after about 5-10 days. The cells are prepared in this “conditioned” media for transfusion at concentrations of less than about 100,000 cells per mL Physiological electrolyte additives may be added. The cell solution is administered intravenously.

In a further method, cells are grown in media for about 5-10 days. This media is then transfused intravenously without cells or given locally to the site of the injury. Further methods involve isolation and/or concentration of stem cell produced factors and/or further refinements of these chemicals and/or compounds.

In embodiments, cell proliferation can be expressed in growth per passage. For example, in disclosed embodiments the isolated MSC can increase in number by 40% per passage, 50% per passage, 60% per passage, 70% per passage, 80% per passage, 90% per passage, 100% per passage, 120% per passage, 150% per passage, 200% per passage, 250% per passage, or the like.

### Activation of MSC

In embodiments, stem cells, for example isolated MSC, can be activated to produce MSC with desired characteristics. For example, MSC can be polarized towards a pro- or anti-inflammatory phenotype depending on the Toll-like receptor (TLR) stimulated. In embodiments, MSC are exposed to inflammatory cytokines to increase expression of adhesion molecules such as VCAM-1 and ICAM-1, which enables MSC to sequester and enhance function of immune cells.

In further embodiments, MSC activated through the TLR 4 pathway increased VCAM-1 and ICAM-1-dependent binding of leukocytes.

### Methods of Formulation of MSC

In embodiments, isolated MSC can be formulated into a pharmaceutically-acceptable composition, for example by using at least one pharmaceutically-acceptable carrier. In embodiments, a pharmaceutically-acceptable carrier means a carrier that is useful in preparing a pharmaceutical composition or formulation that is generally safe, non-toxic, and neither biologically nor otherwise undesirable, and includes a carrier that is acceptable for veterinary use as well as human pharmaceutical use. The pharmaceutically acceptable carrier can comprise, for example, saline solution, phosphate buffered saline (PBS), Ringer's serum, Ringer's lactate serum, lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils.

Disclosed formulations comprise MSC combined with cytokines in the form of a composition, e.g., a pharmaceutical composition suitable for administration to a subject in need of treatment with the same.

Disclosed formulations can be “pre-loaded” into administration devices, for example syringes, prior to use.

Disclosed formulations can be provided as a kit. For example, a disclosed kit can comprise a pharmaceutically acceptable carrier; an isolated population of mesenchymal stem cells; isolated interferons, isolated interleukins, and instructions for using the kit in a method for attenuating an immune response. The cell and cytokine components of the kit can be administered individually, or combined in vitro and subsequently administered as a mixture. The kit also optionally may include a means of administering the composition, for example by injection.

### Methods of Treatment Using MSC

Disclosed embodiments can comprise administration of MSC to treat various conditions and diseases. For example, vesicles derived from placental MSC can be employed for therapeutic uses. In embodiments, the stem cells may be autologous to the subject. If available, autologous stem cells can be beneficial to the subject because they reduce or eliminate the potential for adverse immune responses, e.g., rejection of the stem cells or graft-versus-host disease. Autologous stem cells can be, e.g., stem cells isolated directly from the subject (e.g., MSC), or iPS cells produced from non-stem cells from the subject.

In some embodiments, in cases where autologous stem cells are not available or not indicated for a particular subject, allogeneic stem cells can be used. Allogeneic stem cells should be matched as closely as possible to the subject (e.g., via HLA genotype) in order to reduce the likelihood of rejection or graft-versus-host disease. In other embodiments, the stem cell donor is a first-degree-relative (e.g., parent, sibling, or child) of the subject, which increases the likelihood of finding a closely-matched donor. In yet other embodiments, the stem cell donor can be an extended relative of the subject. In some embodiments, the stem cell donor can be from the same race or ethnic group as the subject. However, certain stem cells can be immune-privileged and can be used allogeneically without matching between the donor and subject.

In embodiments, MSC are used for treatment of human or non-human patients, for example treatment of diseases, conditions, disorders, etc., for example inflammatory, immune-mediated, and musculoskeletal disorders.

Disclosed embodiments comprise methods of treatment of inflammatory disorders. For example, disclosed methods of treatment can comprise treatment of a biological response to stimuli interpreted by the body to have a potentially harmful effect. While after injury, or in certain conditions, inflammation is a normal, healthy response, inflammatory disorders that result in the immune system attacking the body's own cells or tissues may cause abnormal inflammation, which results in chronic pain, redness, swelling, stiffness, and damage to normal tissues. For example, disclosed embodiments can comprise methods of treating vasculitis, Systemic Lupus Erythematosus (SLE; Lupus), Sjogren's Syndrome, scleroderma, myositis, inflammatory arthritis, gout, Ankylosing Spondylitis (AS), Antiphospholipid Antibody Syndrome (APS), asthma, peptic ulcers, tuberculosis, Rheumatoid Arthritis (RA), periodontitis, ulcerative colitis, Crohn's disease, sinusitis, hepatitis, and the like.

Disclosed embodiments comprise methods of treatment of immune-related disorders. These type of disorders can also involve inflammation. Immune system disorders cause abnormally low activity or overactivity of the immune system. In cases of immune system overactivity, the body attacks and damages its own tissues (autoimmune diseases). Immune deficiency diseases decrease the body's ability to fight invaders, causing vulnerability to infections. For example, disclosed embodiments can comprise treatment of, for example, inflammatory bowel disease, lupus, RA, Celiac disease, multiple sclerosis, Guillain-Barre syndrome, Graves' Disease, chronic inflammatory demyelinating polyneuropathy, Hashimoto's thyroiditis, myasthenia gravis, psoriasis, allergies, combinations thereof, and the like.

Disclosed embodiments comprise methods of treatment of musculoskeletal disorders. Musculoskeletal Disorders or MSD are injuries and disorders that affect the human body's movement or musculoskeletal system, For example, disclosed methods of treatment can comprise treatment of injuries or pain to muscles, tendons, ligaments, nerves, discs, combinations thereof, Carpal Tunnel Syndrome, tendonitis, muscle or tendon strain, ligament sprain, Tension Neck Syndrome, Thoracic Outlet Compression, rotator cuff tendonitis, epicondylitis, Radial Tunnel Syndrome, Digital Neuritis, trigger finger or thumb, DeQuervain's Syndrome, degenerative disc disease, ruptured/herniated disc, and the like.

In embodiments comprising treatment of mammals, for example cats, MSC can be used to treat, for example, chronic oral inflammation, chronic kidney disease, diabetes type 1, immune-mediated skin diseases, musculoskeletal disease, Intervertebral disc degeneration, irritable bowel syndrome and other inflammatory or immune-mediated disorders.

In further embodiments, for example embodiments comprising treatment of mammals, for example dogs, MSC can be used to treat, for example, atopic dermatitis, OA, crucial ligament repair, immune-mediated nephropathy, intervertebral disc degeneration, wound healing, irritable bowel syndrome, and other inflammatory and immune-mediated disorders.

In mammals, such as, for example, horses, MSC can be used to treat tendinopathy, ligament tear, recurrent uveitis, immune deficiency, wound healing and other inflammatory and immune mediated disorders. MSC can also be used as energy boost for performance and race horses.

MSC can be administered, for example infused, via any appropriate method, for example subcutaneous, intra-articular, intra-lesional (tendon, ligament, disc), intravenous, intra-peritoneal, or intramuscular administration.

Appropriate MSC dosage can be, for example, 1×10³ cells, 2.5×10³ cells, 5×10³ cells, 1×10⁴ cells, 2.5×10⁴ cells, 5×10⁴ cells, 1×10⁵ cells, 2.5×10⁵ cells, 5×10⁵ cells, 1×10⁶ cells, 2.5×10⁶ cells, 5×10⁶ cells, 1×10⁷ cells, 2.5×10⁷ cells, 5×10⁷ cells, 1×10⁸ cells, 2.5×10⁸ cells, 5×10⁸ cells, 1×10⁹ cells, 2.5×10⁹ cells, 5×10⁹ cells, 1×10¹⁰ cells, 2.5×10¹⁰ cells, 5×10¹⁰ cells, 1×10¹¹ cells, 2.5×10¹¹ cells, 5×10¹¹ cells, 1×10¹² cells, 2.5×10¹² cells, 5×10¹² cells, 1×10¹³ cells, 2.5×10¹³ cells, 5×10¹³ cells, 1×10¹⁴ cells, 2.5×10¹⁴ cells, 5×10¹⁴ cells, 1×10¹⁵ cells, 2.5×10¹⁵ cells, 5×10¹⁵ cells, or more, or the like.

In embodiments, appropriate MSC dosage can be, for example, between 1×10³ cells and 2.5×10³ cells, between 5×10³ cells and 1×10⁴ cells, between 2.5×10⁴ cells and 5×10⁴ cells, between 1×10⁵ cells and 2.5×10⁵ cells, between 5×10⁵ cells and 1×10⁶ cells, between 2.5×10⁶ cells, between 5×10⁶ cells and 1×10⁷ cells, between 2.5×10⁷ cells and 5×10⁷ cells, between 1×10⁸ cells and 2.5×10⁸ cells, between 5×10⁸ cells and 1×10⁹ cells, between 2.5×10⁹ cells and 5×10⁹ cells, between 1×10¹⁰ cells and 2.5×10¹⁰ cells, between 5×10¹⁰ cells and 1×10¹¹ cells, between 2.5×10¹¹ cells and 5×10¹¹ cells, between 1×10¹² cells and 2.5×10¹² cells, between 5×10¹² cells and 1×10¹³ cells, between 2.5×10¹³ cells and 5×10¹³ cells, between 1×10¹⁴ cells and 2.5×10¹⁴ cells, between 5×10¹⁴ cells and 1×10¹⁵ cells, between 2.5×10¹⁵ cells and 5×10¹⁵ cells, or more, or the like.

In embodiments, appropriate MSC dosage can be, for example, not less than 1×10³ cells, not less than 2.5×10³ cells, not less than 5×10³ cells, not less than 1×10⁴ cells, not less than 2.5×10⁴ cells, not less than 5×10⁴ cells, not less than 1×10⁵ cells, not less than 2.5×10⁵ cells, not less than 5×10⁵ cells, not less than 1×10⁶ cells, not less than 2.5×10⁶ cells, not less than 5×10⁶ cells, not less than 1×10⁷ cells, not less than 2.5×10⁷ cells, not less than 5×10⁷ cells, not less than 1×10⁸ cells, not less than 2.5×10⁸ cells, not less than 5×10⁸ cells, not less than 1×10⁹ cells, not less than 2.5×10⁹ cells, not less than 5×10⁹ cells, not less than 1×10¹⁰ cells, not less than 2.5×10¹⁰ cells, not less than 5×10¹⁰ cells, not less than 1×10¹¹ cells, not less than 2.5×10¹¹ cells, not less than 5×10¹¹ cells, not less than 1×10¹² cells, not less than 2.5×10¹² cells, not less than 5×10¹² cells, not less than 1×10¹³ cells, not less than 2.5×10¹³ cells, not less than 5×10¹³ cells, not less than 1×10¹⁴ cells, not less than 2.5×10¹⁴ cells, not less than 5×10¹⁴ cells, not less than 1×10¹⁵ cells, not less than 2.5×10¹⁵ cells, not less than 5×10¹⁵ cells, or more, or the like.

In embodiments, appropriate MSC dosage can be, for example, not more than 1×10³ cells, not more than 2.5×10³ cells, not more than 5×10³ cells, not more than 1×10⁴ cells, not more than 2.5×10⁴ cells, not more than 5×10⁴ cells, not more than 1×10⁵ cells, not more than 2.5×10⁵ cells, not more than 5×10⁵ cells, not more than 1×10⁶ cells, not more than 2.5×10⁶ cells, not more than 5×10⁶ cells, not more than 1×10⁷ cells, not more than 2.5×10⁷ cells, not more than 5×10⁷ cells, not more than 1×10⁸ cells, not more than 2.5×10⁸ cells, not more than 5×10⁸ cells, not more than 1×10⁹ cells, not more than 2.5×10⁹ cells, not more than 5×10⁹ cells, not more than 1×10¹⁰ cells, not more than 2.5×10¹⁰ cells, not more than 5×10¹⁰ cells, not more than 1×10¹¹ cells, not more than 2.5×10¹¹ cells, not more than 5×10¹¹ cells, not more than 1×10¹² cells, not more than 2.5×10¹² cells, not more than 5×10¹² cells, not more than 1×10¹³ cells, not more than 2.5×10¹³ cells, not more than 5×10¹³ cells, not more than 1×10¹⁴ cells, not more than 2.5×10¹⁴ cells, not more than 5×10¹⁴ cells, not more than 1×10¹⁵ cells, not more than 2.5×10¹⁵ cells, not more than 5×10¹⁵ cells, or more, or the like.

In embodiments, MSC can be administered one time, two times, three times every month, or three months, six months or on a yearly basis.

Disclosed methods can also involve the co-administration of bioactive agents with the stem cells. By “co-administration” is meant administration before, concurrently with (e.g., in combination with bioactive agents in the same formulation or in separate formulations), or after administration of a therapeutic composition as described above. As used herein, “bioactive agents” refers to any organic, inorganic, or living agent that is biologically active or relevant. For example, a bioactive agent can be a protein (e.g albumin), a polypeptide, a nucleic acid, a polysaccharide (e.g., heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor-producing virus, a growth factor inhibitor, a growth factor receptor, an anti-inflammatory agent, an antimetabolite, an integrin blocker, or a complete or partial functional sense or antisense gene, including siRNA. It can also include a man-made particle or material, which carries a biologically relevant or active material, for example a nanoparticle comprising a core with a drug and a coating on the core. Bioactive agents can also include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), and analgesics and analgesic combinations. Anti-inflammatory agents may be useful as additional agents to counteract the inflammatory aspects of the fibrotic process.

Combinations, blends, or other preparations of any of the foregoing examples can be made and still be considered bioactive agents within the intended meaning herein. Aspects of the present disclosure directed toward bioactive agents may include any or all of the foregoing examples. In other embodiments, the bioactive agent may be a growth factor. A growth factor is any agent which promotes the proliferation, differentiation, and functionality of the implanted stem cell. Non-limiting examples of suitable growth factors can include, but are not limited to, leukemia inhibitory factor (LIF), epidermal growth factor (EGF), fibroblast growth factor (FGF), insulin-like growth factor (IGF), vascular endothelial growth factor (VEGF), human growth hormone (hGH), platelet-derived growth factor (PDGF), interleukins, cytokines, and/or combinations thereof. Bioactive agents can be a blood-derived supplement containing mixture of growth factors such as platelet lysate.

In embodiments, the bioactive agent can comprise an immunosuppressive agent. An immunosuppressive agent is any agent which prevents, delays the occurrence of, or decreases the intensity of the undesired immune response, e.g., rejection of a transplanted cell, tissue, or organ, or graft-versus-host disease. Preferred are immunosuppressive agents which suppress cell-mediated immune responses against cells identified by the immune system as non-self. Examples of immunosuppressive agents include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art. In other embodiments, bioactive agents can include anti-fibrotic agents including, but not limited to, nintedanib, INT-767, emricasan, VBY-376, PF-04634817, EXC 001, GM-CT-01, GCS-100, Refanalin, SAR156597, tralokinumab, pomalidomide, STX-100, CC-930, simtuzumab, anti-miR-21, PRM-151, BOT191, palomid 529, IMD1041, serelaxin, PEG-relaxin, ANG-4011, FT011, pirfenidone, F351 (perfenidone derivative), THR-184, CCX-140, FG-3019, avosentan, GKT137831, PF-00489791, pentoxifylline, fresolimumab, and LY2382770.

### EXAMPLES

The following non-limiting examples are provided for illustrative purposes only in order to facilitate a more complete understanding of representative embodiments. These examples should not be construed to limit any of the embodiments described in the present specification.

### Example 1—MSC Cell Isolation from Placenta

Placenta samples were collected upon delivery from normal full-term pregnancies. All samples were obtained with written, informed consent in accordance with the ethical committee requirements of the relevant body. The human placenta tissues were processed using mechanical disassociation and enzymatic digestion method. Briefly, placenta tissue was minced into a paste-like consistency and digested in enzymatic mixtures containing 0.4% type II collagenase (Worthington, New Jersey, USA) and 0.01% DNAse (Worthington, New Jersey, USA). Following this, tissues were mechanically dissociated using a hand held cell homogenizer (Hassen Wagger). The single cell suspension is resuspended in MSC complete media containing Dulbecco's Modified Eagle's medium with nutrient mixtures F-12 (HAM) (1:1) with GLUTAMAX-I (Gibco, Invitrogen, USA), 10% foetal bovine serum (Stem Cell Technology Inc., London, UK), 1% Penicillin and Streptomycin (Gibco, Invitrogen), 0.5% Fungizone (Gibco, Invitrogen), 0.1% Gentamicin (Gibco, Invitrogen) and 40 ng/mL basic fibroblastic growth factor (bFGF) (Promega). The single nucleated cells (30×106 cells/T25 culture flask) were cultured in MSC complete media. Primary cultures were incubated for at least a week in a 37° C. humidified 5% CO₂ incubator and non-adherent cells were removed by replacing the media. Upon reaching 70% to 80% confluence, adherent MSC were harvested via trypsinisation (0.05% trypsin-EDTA, Invitrogen, BRL, Canada) for use in downstream experiments.

### Example 2—MSC Cell Isolation from Placenta

Placenta tissue was aseptically collected from eligible donors in a container after birth. All the following steps were performed in a certified clean room under cGMP conditions.

Placenta was washed extensively in rinsing buffer. Placenta was cut in small pieces 1-5 grams. Decidual giant cells were removed by one or combination of steps. Incubation of placenta tissue was performed in Trypsin for 30-90 minutes at 37° C. and 5% CO₂. Filtration is then done using 20, 25 and 30 micron nylon mesh. Gradient separation of the cells using BSA, Percoll, or Ficoll then follows. Differential adhesion was conducted, allowing the quick attaching cells be separated from the nonattached cells that are floating in the media. Placenta tissue was minced for 90 sec or 150 cutting cycles. Enzymatic digestion of placenta was performed at 37° C. using collagenase for 60-180 min with shaking at the rate of 100 to 140 cycle per minute.

Cells were then passed through a sequence of cell strainers (100 micron, 40 micron) and then through a nylon mesh of 20, 25, or 30 micron. In some cases, cells were also be passed through a gradient. Red Blood Cells (RBC) will be removed by RBC lysis buffer 3 min at 4° C. RBC lysis were be neutralized by adding 15-20 times PBS. Cells are then centrifuged at 400 g for 10 min. Next, the cells were cultured at a density of 200-300×10³ per cm² in a culture flask for 5-7 days. In some cases, to remove the remaining giant cells from the mixture, differential adhesion was be applied; cells were allowed to attach for a few hours, and then the floating cells were separated from the attached cells. After 5-7 days, placenta MSC can be harvested from the flask as P0.

### Example 3—MSC Cell Banking

For further expansion of cells beyond P0, MSC were cultured at the density of 50-75×10³ per cm² in an appropriate media.

For scale-up production, in some cases Master Cell Banks were cultured in, for example, bioreactors on microcarriers, or in hollow fiber systems.

Master Cell Banks go through a series of tests to ensure their viability, identity, purity, sterility and functionality. These tests were: mycoplasma testing, microbial testing (aerobic, anaerobic, fungi and yeast), endotoxin testing, karyotype, cell surface marker analysis, in vitro differentiation assay, lymphocyte proliferation assay, Mixed Leukocyte Reaction (MLR), and IDO GE2, TGF-beta, HGF, etc.

MSC should be free from microorganisms and mycoplasma. Placenta MSC should have no maternal cell contamination. Placenta MSC should have normal karyotype and acceptable level of tetraploidy. Viability of the cells throughout the procedure is assessed by an automated cell count system that assess the live/dead cells as well as the average diameter of the cells. Cells are frozen at the concentration of 5-20×10⁶ per vial in a clinical grade cryopreservation media.

In some cases, freshly-thawed cells were prepared for therapeutic use. Frozen cells were thawed in an automated thawing system and after removal of the cryomedia, cells were tested (viability and sterility) and re-suspended in injectable solution in a sterile syringe and shipped to the clinic using a validated shipper. These cells are used within 24 h after shipment. In some indications, frozen cells will be thawed in automated thawing device and after removal of cryomedia the cells will be cultured for 2-3 days to recover the freezing. After 2-3 days, the cells were retrieved from culture by enzymatic digestion and after testing (viability and sterility), freshly cultured cells were resuspended in an injectable solution in a sterile syringe and w shipped to the clinic using a validated shipper. These cells are used within 24 h after shipment.

### Example 4—Intra-articular Administration of Allogeneic Adipose Derived MSCs Reduces Pain and Lameness in Dogs with Hip Osteoarthritis

Cell-based therapies are being studied as potential disease-modifying agents for the treatment of OA both in human and companion animals. Different studies showed that Platelet-rich plasma (PRP), through secretory cytokines, has a positive effect on reducing the inflammation and symptoms of OA. However, due to the short life span of platelets (4-7 days), the effect seems to be transient. Mesenchymal stem cell (MSC) therapy for OA is intriguing for many reasons, but four properties in particular make them appealing: (1) they are a bioactive living system that can work and maintain a lasting and adaptable effect; (2) they are anti-inflammatory and therefore should slow or stop the progress of OA and improve clinical symptoms, (3) they can “home” to the site of injury within the joint to have a more targeted effect, and (4) they have the potential to create new cartilage either directly by differentiation into chondrocytes or by paracrine function to recruit nascent stem cell and progenitor cell populations.

MSCs can be obtained from different sources, such as bone marrow, periosteum, umbilical cord blood, dermis, muscle, infrapatellar fat pad, synovial membrane, and adipose tissue. Among these sources, adipose-derived mesenchymal stem cells (ADSC) are attracting attention as an alternative to the better-studied bone marrow mesenchymal stem cells (BMSC). The described multicentered, randomized and double blinded pilot study was designed to better understand the effect of different doses of allogeneic adipose MSCs and their possible mode of action on improving lameness and mobility in dogs with OA.

This study was conducted to investigate the therapeutic effect of allogenic adipose-derived MSCs on dogs with hip osteoarthritis (OA).

Study Design: Twenty dogs with bilateral osteoarthritis of the coxofemoral (hip) joint, diagnosed by a veterinarian through physical examination and radiographs, were randomly allocated in four groups. Group 1 served as placebo control (Sham) and injected with saline (n=4). Group 2 were injected with a single dose of 5 Million MSC (n=5). Group 3 received a single dose of 25 Million MSC (n=6) and Group 4 received a single dose of 50 Million MSC (n=5).

Protocol: Two weeks prior to and during the study, the dogs were taken off of NSAIDs (Non-teroidal Anti-Inflammatory Drugs) and corticosteroids. At the day of administration, dogs were examined by a veterinarian (vet-1) who was blinded from the study for lameness score. Radiographic images were taken to document the stage of OA prior to the study. The dogs were then subjected to gait analysis to document the weight bearing and length and time of the strides during walk and trot. A second veterinarian, (vet-2), then performed the intra-articular injections. Prior to administration of cells or saline, a sample of synovial fluid and peripheral blood were collected to look for inflammatory and anti-inflammatory biomarkers. Physical exam, lameness score, gait analysis, synovial fluid and blood samples were taken again during the 5, 30 and 90 days follow ups. In addition, a CBPI (Canine Brief Pain Inventory) score form was provided to the pet owners to record the severity of pain and mobility according to their observation (Form 1). The data was then analyzed by vet-1 who was blinded from the study.

Results: Intra-articular administration of allogeneic MSC into multiple joints did not result in any serious adverse events. The average lameness score of the dogs in placebo control group did not show improvement after 30 or 90 days of intra-articular administration of saline. However, the average lameness score of the MSC-treated dogs was improved at both time points. Overall, seventy-eight percent of the dogs that received various doses of MSC showed improvement in the lameness score 90 days after intra-articular administration of MSC. ELISA tests on peripheral serum showed that all the dogs with improved lameness in the placebo group and most of the dogs treated with MSC had an elevated serum IRAP 30 days after intra-articular administration. None of the dogs in the placebo group that showed lameness improvement had elevated IL-10 in peripheral blood. However, about seventy percent of the dogs (4/6) with improved lameness that were treated with various doses of MSCs showed elevated serum IL-10. The level of serum IL-10 after intra-articular administration of MSCs seems to correlate with the number of cells administered.

Conclusions: Intra-articular administration of allogenic adipose derived MSC is safe and improved lameness scores in dogs associated with OA in the hip joint. Our results showed that all doses of MSC were effective, however there was a trend indicating that more cells result in more improvement. The improved lameness effect was present at least for 90 days. Serum levels of IL-10 was increased in a majority of the dogs that received MSCs and also had improved lameness. Thus, IL-10 seems to be a good indicator of the lameness improvement due to cell therapy. All the dogs that showed improvement in their lameness score had an elevated serum level of RAP, thus, the serum level of RAP seems to be a good indicator of lameness improvement irrespective to treatment.

### MATERIALS AND METHODS

### Donor Eligibility and Collection of Adipose Tissue

For this study, at a local veterinary clinic, a five month old, 72 pound, healthy female mastiff dog was selected as a tissue donor. Adipose tissue that is normally discarded was collected from mesometrium fat and ovarian fat pads during a routine ovariohysterechtemy surgical procedure under general anesthesia. The tissue was immediately transferred to a sterile tissue collection container in sterile cold PBS and was sent to VCT manufacturing facility in a validated shipper. The validated shipper is constantly monitored with an internal data logger and maintains the temperature of the tissue in a regulated environment between 2-8° C. for up to 24 hours. The patient recovered as per normal procedure without any adverse events.

### Production of MSCs in cGMP Condition

Upon arrival, the box containing adipose tissue was transferred to our production facility. The production facility consists of an ISO class 7 certified clean room containing ISO class 5 certified biosafety cabinets for adipose tissue SVF isolation and MSC culture. Adipose tissue processing was done according to VetCell Therapeutics Standard Operating Procedures (VCT-SOP). Cells were quality control-tested for the number of cells isolated, viability, sterility, and environmental monitoring the biosafety cabinet. Cell counts were performed an automated cell count (Chemometec NC-200 Nucleocounter). Cells were then cultured up through passage 2 (P2) according to VCT-SOP for culture of canine adipose derived MSCs. After culturing, the MSC were cryopreserved and suspended in Cryostor CS10 cryopreservation solution (BioLife Solutions) in a controlled-rate freezer. As part of the requirement of the batch release, all MSC batches undergo QC testing including tests for sterility (Gram +, Gram (−), fungi, yeast), endotoxin, mycoplasma, MSC-specific cell surface marker expression, post-production adventitious agent screening, mixed leukocyte reaction (MLR) with PGE2 and IDO cytokine measurements, trilineage differentiation to adipocytes, osteocytes and chondrocytes, and karayotype. For this study, only one batch of MSC was used.

### MSC Storage and Shipment

After cryopreservation, the MSC were transferred to a vapor-phase liquid nitrogen dewar at cryogenic temperature (≤150° C.). The MSCs remain in this condition until they are thawed for preparation of therapeutic dosing. The temperature of the dewar was constantly monitored and recorded to ensure the MSC remain at cryogenic temperature for the duration of their storage.

### Preparation of Therapeutic Dose of MSC

Preparation of a therapeutic dose was performed in an ISO class 5 biosafety cabinet at VCT-Asia satellite lab. The MSC were thawed in a ThawStar automated cell thawing system (Asterobio). After thawing, the cells were quickly removed from the cryopreservation medium by dilution, centrifugation, and resuspension in a DPBS-FBS buffer. After counting, the appropriate dosage of cells were washed with DPBS, centrifuged again, and resuspended in DPBS. The cells were then loaded into a sterile syringe and placed in a sterile syringe sleeve identifying the name and ID number of the recipient. These syringes were then either placed in a refrigerator (4° C.) first before being transferring into a cooled (2-8° C.) validated shipping system for transfer to the clinic; the syringes were also transferred directly into the validation shipping system without the temporary storage in the refrigerator. Syringes were transferred into the validated shipper no more than 20 minutes after being prepared, and delivered to veterinary clinics no more than 4 hours after being prepared.

### Administration of Cells into Coxofemoral Joints

Some patients were pre-medicated with acepromazine and butorphanol, and all patients were placed under general anesthesia with propofol, intubated and maintained on a mixture of isofluorane and oxygen. Patients were positioned in lateral recumbency, with the joint of interest dorsal (facing up). The dorsal hind limb was kept in a normal standing anatomical position and allowed to hang off of the edge of the surgery table to help to open the joint space. The injection site was prepared as for aseptic surgical procedure by clipping the hair, scrubbing with chlorhexidine surgical scrub, followed by a final alcohol scrub. The surgeon was sterile scrubbed, gowned and gloved. A 22 gauge spinal needle was directed perpendicular to the skin, just dorsal to the greater trochanter and into the coxofemoral joint space. Commonly, external rotation and distal traction was applied to the limb to aid entry into the coxofemoral joint space. Entry into the joint space was confirmed by “surgeon-feel” of the slight “pop” through the joint capsule and/or by aspiration of synovial fluid with a leuer lock 3 ml or 5 ml syringe. In some patients, no synovial fluid was aspirated. The 1 ml leuer lock syringe containing 0.6 ml of product was aseptically removed from its sterile sleeve, the syringe cap was removed, 0.2 ml of air was introduced and the syringe was inverted 10 times to mix the cells. Air was then expelled, and the product syringe was ready for use. Keeping the needle in its place within the joint, the 3 ml or 5 ml syringe was removed, and the 1 ml product syringe was securely attached to the hub of the needle. The entire 0.6 ml was slowly injected within the joint space over five seconds. The syringe and needle were then withdrawn from the patient and firm pressure was applied over the puncture site for thirty seconds, followed by taking the limb through a full range of motion a few times. This procedure was repeated on the contralateral coxofemoral joint.

### Patient At-home Care Post Intra-articular Injection

Owners were instructed to try and keep the patient quiet and cage-rested. Patients were allowed to perform slow, well-controlled 5-10 minute leash walks up to 2 times a day for the first 7 days post injection. After this, owners were instructed to perform slow, well-controlled leash walks for no more than 30 minutes twice daily for the duration of the study. Owners were instructed not to allow any running, jumping, playing, stairs, or over-exertion. Patients were not allowed to start any additional rehabilitation programs and were instructed to maintain a fairly constant weight for the 3-month study duration. Over-exertion and any dog-dog interactive play or going up and down stairs and jumping on and off of any furniture was avoided. No extra rehabilitation program was allowed during this time.

### Study Design

In total, 20 dogs were enrolled in two veterinary clinics in Hong Kong. Sixteen dogs were injected with various doses of CAD-MSC intra-articularly. Five dogs received 5 million cells per joint, six dogs received 25 million cells per joint and five dogs received 50 million cells per joint. The majority of dogs received injections into both hip joints and only a few dogs had 1-2 additional joints injected at the same time. Four dogs in the Placebo group were injected with placebo media (sterile saline) lacking MSC intra-articularly (Table 1).

The study was randomized and double blinded. Each dog was assigned a number. Owner and veterinarian-1 (Vet-1) were not aware of what was injected. Veterinarian-2 (Vet-2) performed the intra-articular injection procedure and knew what each patient received, and this information remained confidential to Vet-2 and VCT researchers only.

The following procedure was applied to all patients: VCT-Asia technician supplied VCT syringe in shipping container on each surgery day. Each syringe has been assigned randomly to denote treatment vs. placebo and this has been documented for each patient by the VCT-Asia and VCT technicians and Vet-2 (surgeon). Vet-1 was the pre- and post-surgery consultant and diagnostician for each individual patient. Vet-2 has performed the intra-articular injections for each patient. Daily calorie intake was allowed to be modified so the patient maintained a constant weight throughout the study. At the end of the study, the pets that were given placebo injections were given the option to receive stem cell treatment.

### Patient Enrolment Criteria

Inclusion Criteria: Subject must be a domestic canine patient. Veterinarian and owner must sign and agree to the terms and risks associated with the study (double-blinded study, long term follow up, no other intervention, etc.). Must be at least 1 year old. Must weigh at least 9 kg (19.8 lbs.). Any breed is acceptable. Either sex is acceptable. Must be diagnosed with OA of one or both of the hip (coxofemoral) joints by a licensed veterinarian. Patients must have noticeable lameness, limited range of motion, and evident pain on palpation/manipulation. Radiographs must show evidence of arthritic changes. Must have at least 1 month of symptoms associated with OA. Must have undergone at least 1 month of medical and/or physical therapy/cage rest management with little or no improvement. Must be deemed fit and healthy to the best of our knowledge for anesthesia and surgery. Patient must have a BCS of 7/9 or less and must maintain a consistent weight throughout the study. Patient must have a minimum of 1 week no treatment with non-steroidal anti-inflammatories (NSAIDS) prior to the study and no NSAIDS can be started throughout the study. Any additional treatments must be ceased a minimum of 1 week prior to the start of the study. No extra treatments can be performed during the study such as, but not limited to Adequan injections, other joint injections, other pain medications, physical therapy, acupuncture, low level laser, etc. Tramadol use is acceptable for 2-3 days post arthrocentesis to help alleviate acute pain but may not be continued past 3 days post arthrocentesis. Patient must have proven ability to produce good walks on Gait4Dog walkway with reliable and repeatable results.

Exclusion criteria: Must not have any additional known significant illness, infection or disease (especially cancer, IMHA, CCL rupture, joint luxation, bone fractures, IVDD, Lyme disease, etc.). No recent surgery on affected area. If surgery was performed previously, it must have been at least greater than 1 year since surgery. If a total hip replacement procedure has been performed, this joint cannot be used for the study. The contralateral joint may still be used.

### Clinical and Paraclinical Examinations

Lameness assessment and scoring was done by orthopedic exam with range of motion and pain assessment at walk and trot (Table 2).

TABLE 2
Distribution of Patients involved in this study among Different Treatment Groups

| | Patient | | | | | | | | Number of | |
|---|---|---|---|---|---|---|---|---|---|---|
| Treatment | ID | Age | | Weight | | X-rays | X-rays | Joints | Joints | Total |
| Group | Number | (yrs) | Sex | (kg) | Breed | before | after | Injected | Injected | Cells ×10⁶ |
| Placebo | 1 | 8 | F | 21.5 | German | Yes | (90 | Hips | 2 | 0 |
| | | | | | Shephard | | day | | | |
| Placebo | 2 | 10 | F | 46.1 | Swiss | Yes | (90 | Hips | 2 | 0 |
| | | | | | Mountain | | day) | | | |
| | | | | | Dog | | | | | |
| Placebo | 3 | 13 | F | 22.6 | Mongrel | Yes | No | Hips | 2 | 0 |
| Placebo | 4 | 7½ | F | 10.8 | Bichon Frise | Yes | No | Hips | 2 | 0 |
| 5M | 1 | 11½ | F | 34 | Golden | Yes | (90 | Hips | 2 | 10 |
| | | | | | Retriever | | day) | | | |
| 5M | 2 | 13 | F | 25.7 | Labrador | Yes | (90 | Hips | 2 | 10 |
| | | | | | Retriever | | day) | | | |
| 5M | 3 | 14 | M | 21.9 | Golden | Yes | No | Hips, | 4 | 20 |
| | | | | | Retriever | | | elbows* | | |
| 5M | 4 | 11 | M | 31.8 | Mongrel | Yes | No | Hips | 2 | 10 |
| 5M | 5 | 1 | M | 40 | Bernese | Yes | No | Hips | 2 | 10 |
| | | | | | Mountain | | | | | |
| | | | | | Dog | | | | | |
| 25M | 1 | 14 | M | 22.5 | Labrador | Yes | No | Hips | 2 | 50 |
| | | | | | Retriever | | | | | |
| 25M | 2 | 11 | F | 29.5 | Golden | Yes | (90 | Hips | 2 | 50 |
| | | | | | Retriever | | day) | | | |
| 25M | 3 | 13 | F | 11.8 | Cocker | Yes | No | Hips, | 4 | 100 |
| | | | | | Spaniel | | | stifles* | | |
| 25M | 4 | 11 | M | 43.6 | Labrador | Yes | (90 | Hips | 2 | 50 |
| | | | | | Retriever | | day) | | | |
| 25M | 5 | 14 | M | 29.3 | Golden | Yes | No | Hips, | 4 | 100 |
| | | | | | Retriever | | | stifles* | | |
| 25M | 6 | 4 | M | 25.3 | Bulldog | Yes | No | Hips | 2 | 50 |
| 50M | 1 | 12 | F | 21 | Mongrel | Yes | No | Hips | 2 | 100 |
| 50M | 2 | 11½ | M | 29 | Golden | Yes | (90 | Hips | 2 | 100 |
| | | | | | Retriever | | day) | | | |
| 50M | 3 | 3 | M | 46.7 | Caucasian | Yes | No | Hips, | 3 | 150 |
| | | | | | | | | R-stifle* | | |
| 50M | 4 | 4 | F | 23.3 | Bulldog | Yes | No | Hips | 2 | 100 |
| 50M | 5 | 14 | F | 23.7 | Golden | Yes | (90 | Hips | 2 | 100 |
| | | | | | Retriever | | day) | | | |

*Subjects received additional injections in other joints
No: No X-ray (Radiograph) data is available
5M: 5 × 10⁶ cells
25M: 25 × 10⁶ cells
50M: 50 × 10⁶ cells

Lameness score was done prior to injection, Day 0 (day of injection), Day 5 after injection (no running), Day 30 after injection and Day 90 after injection. Radiographs were taken by a licensed veterinarian to confirm the presence of degenerative joint disease prior to injection and at Day 90 after injection in some patients. Images were taken from three different views: ventro-dorsal extended view, ventro-dorsal frog leg view and right lateral view. Synovial fluid was to be collected at Day 0 (day of injection) and Day 90 after injection, but some patients did not result in synovial fluid collection. Synovial fluid was to be used for standard protein, blood cell quantifications, bacteria, color, viscosity and ELISA tests. Peripheral blood was taken prior to cell injection, at Day 0 (day of injection), Day 5 after injection, Day 30 after injection and Day 90 after injection for anti-inflammatory and immune modulatory biomarkers. Owner pre- and post-Canine Brief Pain Inventory (CBPI) form was used to assess the owner's perception of the animal's pain and mobility at home at Day 0 (day of injection), Day 5 after injection, Day 30 after injection, and Day 90 after injection. In addition, veterinarian pre- and post-assessment forms were used to document patient lameness scoring and pain score at Day 0 (day of injection), Day 5 after injection, Day 30 after injection and Day 90 after injection.

### Cytokine Measurement in Canine Plasma

Levels of IL-1RA were measured using a Kingfisher Biotech ELISA kit (St. Paul, MN), following the manufacturer's recommendations as modified by S. S. Huggins et al. Serum concentrations of canine interleukin-1 receptor antagonist protein in healthy dogs after incubation using an autologous serum processing system. Briefly, Nunc-Immuno MaxiSorp 96-well plates (Nalge Nunc, Rochester, NY) were coated with 100 μL of 2 μg/mL capture antibody. A standard curve ranging from 5,000 pg/mL to 19 ng/mL was prepared. Samples were diluted 1:8 with reagent diluent before plating, and 100 μL of standards and samples were run in duplicate. The detection antibody was diluted to 400 ng/mL, and 100 μL of this was added to each well. ELISAs were read at 450 nm, with 540 nm background subtraction, on a Synergy HT Multi-Mode microplate reader with Gen5 software (Biotek, Winooski, VT). Concentrations were calculated on a 4-parameter non-linear regression curve. Level IL-10 was measured with a canine cytokine magnetic bead panel (CCYTOMAG-90K, Millipore, Billerica, MA). Samples were diluted 1:2 with assay buffer and run according to the manufacturer's instructions. Standard curves from 50,000 pg/mL to 12.2 pg/mL were prepared and run at the same time. Plates were read on a Luminex 200 instrument using Xponent software (Luminex Corporation, Austin, TX). Concentrations were calculated on a 5-parameter logistic curve.

### RESULTS

### Distribution of Sex, Age and Weight of the Recipients in Different Groups

Results were presented as mean±standard deviation. For side by side comparison of groups, the data was analyzed with two-sample t-test and for analysis of variance ANOVA was used. P<0.05 was considered statistically significant.

In total, 20 subjects were enrolled in this study. From four subjects in the placebo group, all were female with the average age of 9.6 years and average weight of 25.2 kg. Three of the dogs in the 5M group were male and two were female. The average age was 10 years and average weight was 30.68 kg. From six subjects in the 25M group, three were male and three were female, with an average age of 11.2 years and average weight of 27 kg. From 5 subjects in the 50M group, two were male and three were female, with the average age of 8.9 years and average weight of 28.7 kg (Table 3).

TABLE 3
Sex, age and weight of the dogs in each treatment group

| | | Number of | | Number of | | Average | Average |
|---|---|---|---|---|---|---|---|
| Treatment | Total | Females | | Males | | Age | Weight |
| Group | Number | (%) | | (%) | | (Yrs) | (kg) |
| Placebo | 4 | 4 | (100) | 0 | (0) | 9.6 | 25.2 |
| 5M | 5 | 2 | (40) | 3 | (60) | 10 | 30.68 |
| 25M | 6 | 3 | (50) | 3 | (50) | 11.2 | 27 |
| 50M | 5 | 3 | (60) | 2 | (40) | 8.9 | 28.7 |

5M: 5 × 10⁶ cells
25M: 25 × 10⁶ cells
50M: 50 × 10⁶ cells

### Effect of Age, Sex and Severity of Lameness on Therapeutic Response

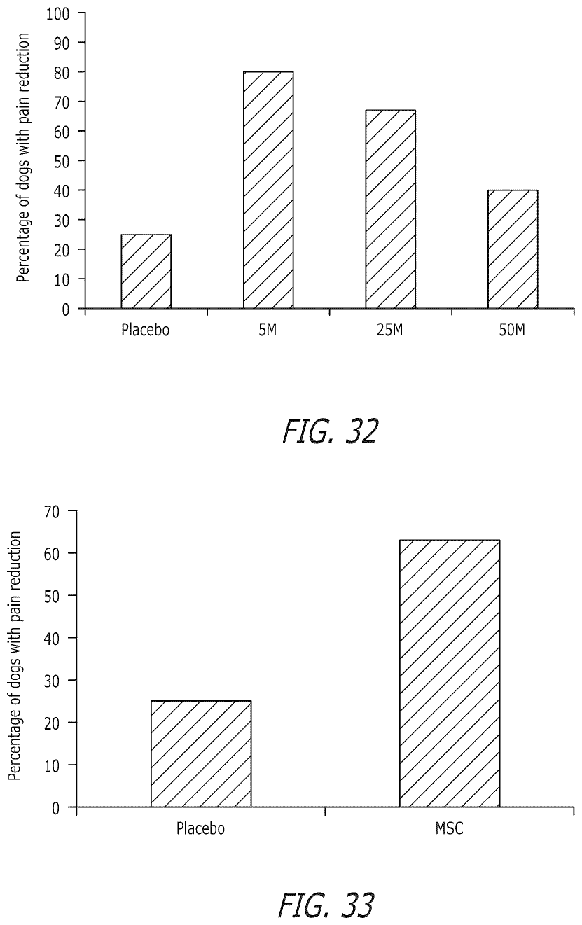

Seventy five percent (3/4) of the dogs under 10 years old had improved lameness following MSC administration. Only seven of the twelve (58.3%) dogs over 10 years old showed improved lameness after cell therapy. Although this is a small and uneven sample size, this indicates that younger dogs had a higher success rate as compared to the older dogs. Five of eight (62.5%) female dogs treated with various doses of MSCs had improved lameness. Also, five of eight (62.5%) male dogs treated with MSCs showed lameness improvement. This indicates that sex of the recipients had no influence on the therapeutic effect of MSCs. Interestingly, (6/6) of the dogs with low to moderate lameness responded to MSC administration and showed improved lameness scores. However, only 5/7 (71.4%) of the dogs with severe lameness responded to MSC administration and showed improved lameness scores. This indicates that cell therapy at the earlier stage of osteoarthritis result in more successful recovery. Owner assessment evaluations also supported lameness scores (FIGS. 32 and 33).

### Effect of Intra-articular Administration of MSCs on Lameness and CBPI Scores

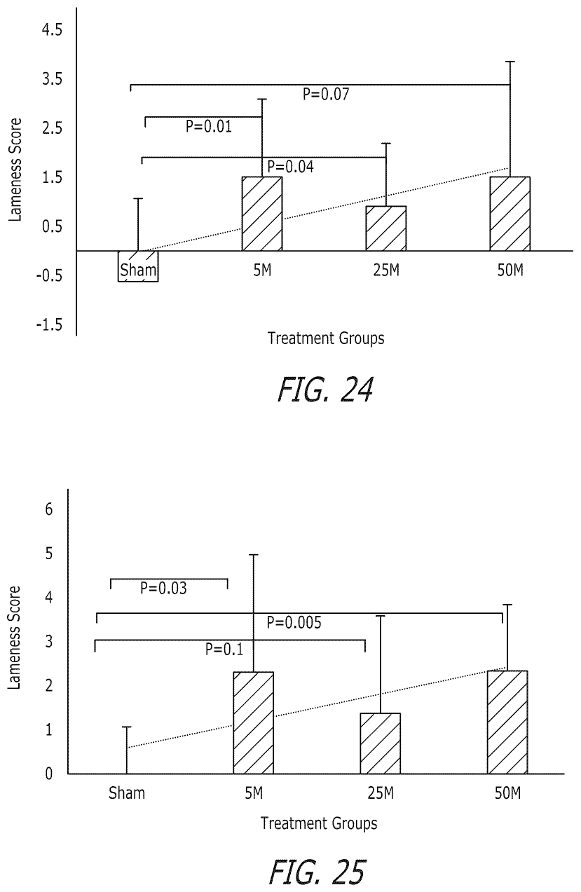

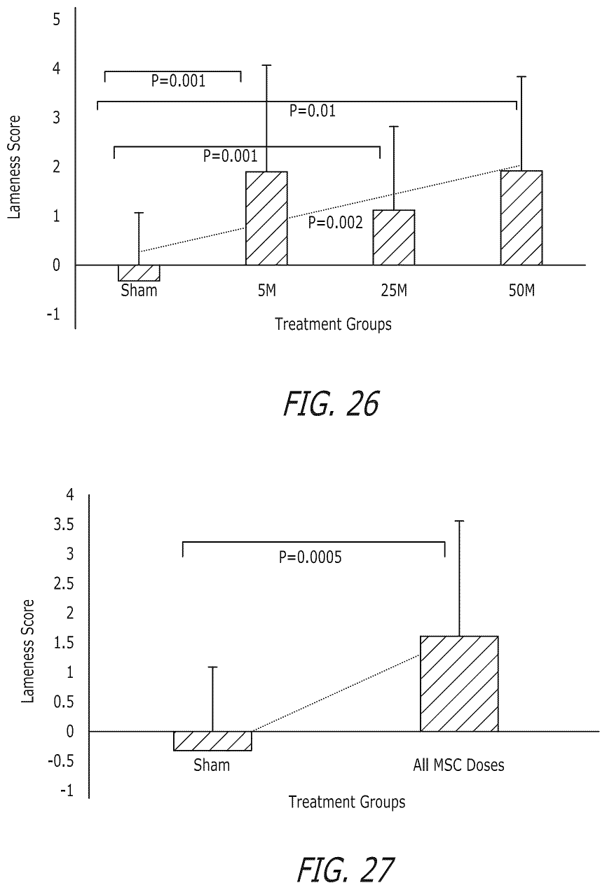

Dogs injected with 5 and 25 million MSC/joint showed significant improvement in their lameness scores during the first 30 days after injection (P<0.05). Dogs injected with 50 million MSC/joint also had improved lameness scores as compared to the placebo, but due to a small sample size and large standard deviation the difference was not significant (P=0.07). Overall, there was a trend indicating that more cells resulted in more lameness improvement. The dogs in the Placebo group did not show any lameness improvement during the first 30 days (FIG. 24). Dogs injected with 5 (P=0.03) and 50 (P=0.005) million of MSCs/joint showed significant improvement in their lameness score after 90 days. Dogs injected with 25 million MSC/joint also had improvement, but due to a large standard deviation the difference was not significant (P=0.1). Similar to the 30-day data point, there was a trend showing more cells resulted in more lameness improvement. The dogs in the Placebo group did not show any lameness improvements after 90 days (FIG. 24). When all data from both 30-day and 90-day improvements from the placebo group and different MSC doses were combined, the results showed that all MSC doses significantly (P≤0.01) improved lameness during the 90 days study as compared to the Placebo group. A trend was observed showing more cells resulted in significantly (P=0.002) more lameness improvements (FIG. 26). Irrespective of MSC dose, when compared against the Placebo group, the results clearly showed that a single intra-articular administration of MSC significantly (P=0.0005) improved lameness during the 90-day study period (FIG. 27). For canine brief pain inventory (CBPI) owner assessments, all MSC doses resulted in significant pain or interference reduction as compared to placebo (FIG. 32). Importantly, only 25% (1/4) of the dogs in the placebo group showed significant pain or interference reduction, while 63% (10/16) of the dogs that received MSC had significantly less pain or interference as compared to the placebo group (P<0.05) (FIG. 33).

### Serum Levels of IRAP and IL-10 in Patients with Improved Lameness

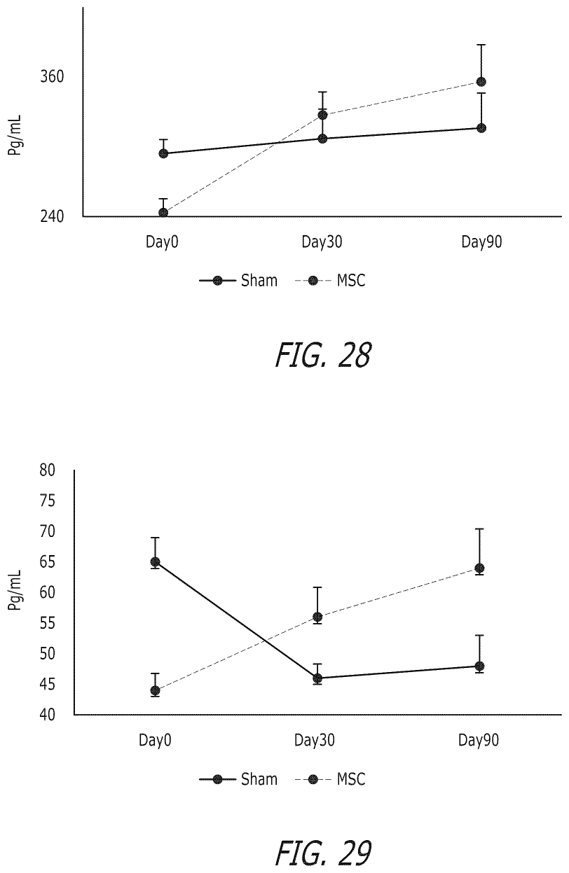

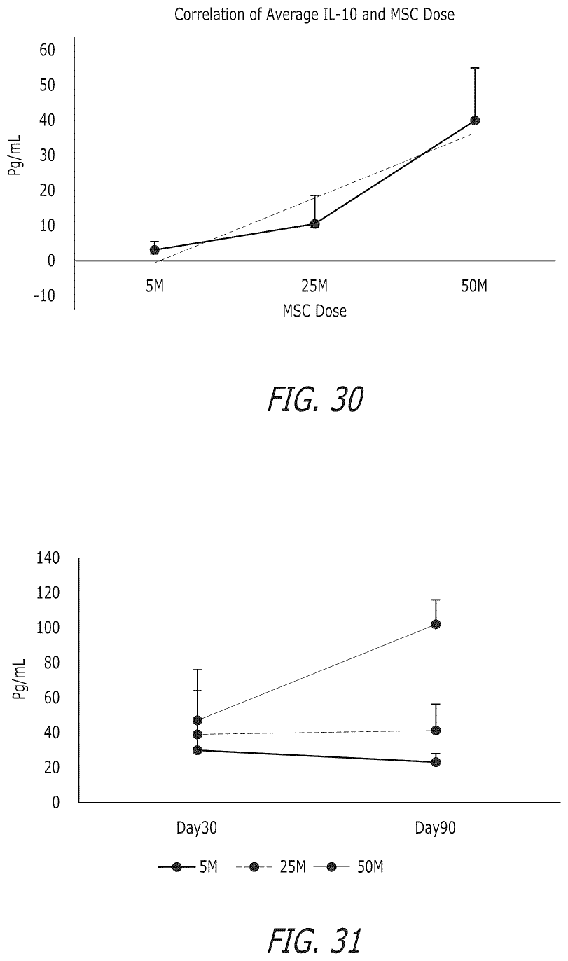

The results collected from this group of dogs showed that all the dogs that had lameness improvements after 30 days, regardless whether they were in the Placebo or MSC-treated groups, had an elevated blood IRAP. This indicates that elevated blood IRAP 30 days after intra-articular injection is a good indication of improvement of lameness irrespective to the treatment group (FIG. 28). The data also shows that, contrary to elevated IRAP, IL-10 was not elevated in the plasma of any Placebo dogs that had improvement in lameness scores. However, 67 percent of dogs (4/6) with improved lameness that were treated with various doses of MSCs showed elevated serum IL-10. This indicates that an elevated level of IL-10 in peripheral blood, 30 days after MSC administration might be a good indicator of the improved lameness due to cell therapy (FIG. 29). Based on this result, peripheral serum IL-10 levels might be a good indication of effectivity of MSC therapy for OA. Furthermore, our data showed that the level of IL-10 in peripheral blood 90 days after intra-articular administration of various doses of MSC increases in a dose dependent manner (FIG. 30). Serum IL-10 levels in patients that received low dose (5M) of MSC declined after 30 days of injection and the IL-10 level remained the same in dogs that received 25M MSC. Interestingly, serum IL-10 continues to increase between day 30 and day 90 only in patients that received our highest dose (50M) of MSC (FIG. 31).

### Discussion

Our double-blinded, placebo-controlled study clearly showed that a single intra-articular administration of allogeneic canine adipose-derived MSC improved the lameness score and increased mobility of dogs with OA in the hip joint. The effect was observed in some dogs as early as 5 days after intra-articular administration of MSC and it was prominent after 30 days of treatment and continued to improve lameness up to at least 90 days (as the last point of observation in this study). In addition to lameness scores performed by veterinarians, in this study we also collected information from pet owners and asked for their opinion about their pet mobility and pain, utilizing the validated CBPI. Data collected from CBPI forms was in-line with the veterinarian lameness scores, indicating that improved pet mobility and reduced pain during daily activity as observed by the owner was consistent with lameness scores performed by the veterinarian. It should be noted that the dogs enrolled in this study did not receive any anti-inflammatory medication or any additional supplements or therapies two weeks before and for the duration of the 90 day study period.

Multiple factors can influence the outcome of the cell therapy including donor, recipient, dosing, cell therapy formulation, route of administration and operators. The quality and origin of the donor tissue has a great impact on the quality of cells obtained from that tissue. Previous studies showed that clinical improvements in signs of canine OA can be achieved using autologous stromal vascular fraction (SVF) of adipose tissue or autologous adipose derived MSC. Our observations collecting SVF from numerous dogs for autologous therapy, and also derivation of MSC from canine adipose tissue for autologous administration revealed that these stem cell products vary greatly between individuals, meaning that only from a subset of patients can a sufficient number of good quality MSC can be obtained (unpublished data). The age of the donor also has a significant impact on the quality of MSC derived from the donor tissue. In this study we used adipose tissue from a young healthy donor (5 months old) to derive MSC for allogeneic use. It has been demonstrated that with aging, the number of mesenchymal stem cells in the body diminishes and this is the time that most of the patients develop osteoarthritis. Therefore, availability of a good quality allogeneic mesenchymal stem cell product from a young healthy donor for these patients is essential.

Although the MSC used in this study were derived from a female donor, both male and female recipients responded equally to the MSC administration. This indicates that the sex of the donor cells does not affect the clinical efficacy of the mesenchymal stem cells in different sex groups. While there is no evidence that adipose derived MSC from female donors are superior to those derived from male, superiority of bone marrow derived MSC from female donors for reducing neonatal hyperoxemia-induced lung inflammation and vascular remodeling is reported. This superiority is thought to be due to different cytokine secretory ability of female MSC. Interestingly, Lipopolysaccharide (LPS) provoked significantly more VEGF production in female MSC versus male MSC supporting sex dimorphism in activated mesenchymal stem cell function.

Dose of the cells, number of injections, injection schedule and site of administration also can change the outcome of the stem cell therapy. To date, not much information is available as to how many cells are needed to exert a therapeutic effect on a patient with hip osteoarthritis. Different doses of MSC were tested in this study. Interestingly all doses were effective in reducing pain and increasing mobility for a similar period of time. There was a trend indicating that more cells resulted in a better effect, however, this observation needs to be proven in a larger scale clinical study enrolling more dogs in different treatment groups. A phase I/II multicenter randomized placebo-controlled clinical trial with thirty human patients with knee OA revealed that administration of higher dose (100 million) of autologous BM-MSC has a more profound and sustainable effect than lower dose (10 million). Another study using human adipose derived MSCs in a mouse model of OA showed that a higher dose of 50 million cells was more effective.

Most of the studies so far, including this study, used a single intra-articular dosing strategy. More studies are needed to investigate whether multiple administrations can improve the therapeutic effect. Some studies using a combination of intra-articular and intravenous administration showed therapeutic benefit, in particular for patients with polyarthritis. Repeated intra-articular administration of canine allogeneic adipose derived MSC to healthy dogs is reported to be safe. We also found that a single intra-articular administration of MSC is safe and did not cause sever adverse reactions. Few dogs showed transient flare after intra-articular administration of MSC, especially after higher doses, which disappeared with rest and tramadol for 2-3 days. No other adverse event happened after intra-articular administration of MSC.

In addition, the quality of the MSC manufacturing, skill of operators during preparation and administration of cells, the viability and functionality of cells after cryopreservation, culturing the thawed cells and preconditioning prior to administration as well as the adjuvant added to cells for resuspension and injection may all affect the efficacy of cell therapy. Our MSC were produced under cGMP conditions and our product consistently met rigorous specifications and maintain high quality in terms of cell surface marker expression, viability, proliferation, sterility, karyotype and endotoxin screening. In addition, we used freshly thawed cells and resuspended them in normal saline for intraarticular administration. We used a controlled-rate freezer system for cryopreservation and a clinical grade cryoprotectant in our study and cells used in this study consistently had viability of above 95% after thaw. We also found that the cell surface marker profile of the canine MSC did not change after cryopreservation (data not shown). A recent study showed that intra-articular administration of freshly thawed allogeneic adipose derived MSC in a DMSO-based cryopreservation medium was also effective in improving pain and lameness of dogs with osteoarthritis. While some investigators believe that MSC need to be cultured and preconditioned after cryopreservation and prior to administration for therapeutic use, others believe that MSC functionality is not changed after cryopreservation. Our data also clearly shows that freshly thawed and washed cells are functional as they improved lameness and pain in dogs with osteoarthritis.

The addition of platelet rich plasma (PRP) to mesenchymal stem cells can act as an adjuvant and enhance their therapeutic effects. There are many advantages with using platelet concentrates as an adjuvant for stem cell therapy. When used as medium supplement, PRP has been shown to promote the growth of adipose derived MSC and maintain their differentiation potential. Furthermore, the antimicrobial and anti-inflammatory properties of PRP might represent a valuable adjunct to the enhancement of tissue regeneration. Wound healing and neovascularization in a porcine model were enhanced only when MSC were topically administered along with PRP, and these effects were largely attributed to the large amount of growth factors found in PRP. The regenerative potential of platelet concentrates and adipose derived MSC on hard and soft tissues has been explored considerably during the last decade. Preclinical studies in animal models have confirmed the synergistic effects of adipose derived MSC and PRP in the healing process of wounds and in osteoarthritis. PRP may also help improve MSC viability and recovery from the therapeutic transport vessel and it may act as a scaffold to help keep the MSCs from migrating out of the affected joint after intra-articular administration.

The age and severity of the degenerative joint disease in patients with OA can negatively impact the clinical outcome of a stem cell treatment protocol. Our study showed that younger patients had a better response to MSC therapy suggesting that the overall health and condition are significant factors determining the response to MSC therapy. All of the dogs with low to moderate lameness responded to MSC administration and showed improved lameness scores. However, only about 70% of the dogs with severe lameness responded to MSC administration and showed improved lameness scores. This indicates that cell therapy at the earlier stage of osteoarthritis results in a more successful therapeutic effect. A similar observation has been reported in a recent study using allogeneic adipose derived MSC with a larger population of dogs with osteoarthritis.

Among many candidate biomarkers we tested in this study, two markers (IRAP and IL-10) showed an interesting relationship with lameness improvement. All the dogs in the Placebo group as well as the majority of the dogs treated with MSC that also had improved lameness had an elevated blood IRAP 30 days after intra-articular administration. This indicates that elevated blood IRAP 30 days after intra-articular injection is a good indication of improvement of lameness irrespective to the treatment group. Increasing the blood level of IRAP in OA patients might be a natural response of the body to arthritic condition. It has been well documented that patients with OA have elevated level of IRAP in their blood. The amount of IRAP in the systemic serum may be directly linked to the amount of lameness improvement. Anti-inflammatory properties and utilization of IRAP for treatment of OA in dogs and horses are reported [60-61]. Our data for the first time showed a positive correlation between the serum level of IL-10 in dogs and their response to MSC administration. Interestingly, the level of IL-10 was maintained at a high level even 90 days after a single intra-articular dose of MSC. More importantly, there was more IL-10 in the serum of dogs that received higher doses of MSC indicating that more stem cells resulted in a more profound IL-10 enhancement. How intra-articular administration of MSC results in a higher elevation of IL-10 is unclear. Our MSC produce IL-10 and thus they could release IL-10 in the synovial fluid and diffuse into peripheral blood following administration. Alternatively, MSC can interact with host immune cells and induce IL-10 production.

In summary, this double-blinded placebo-controlled pilot study clearly showed that a single intra-articular administration of allogeneic canine, adipose-derived MSC was safe, improved the lameness score and increased mobility of dogs suffering from hip osteoarthritis. The effect of MSC on lameness improvement seemed to be dose dependent, however as few as 5 million MSC were effective. Lameness improvement was seen as early as 5 days after MSC administration and continued to improve during the course of the 90-day study period. Our data also provided some insight as to how MSC may exert their anti-inflammatory effect in patients with osteoarthritis by systemic elevation of anti-inflammatory cytokines. A subsequent study with more numbers of patients in each group is needed to confirm the results of this pilot study.

### Example 5—Treatment of Tendinopathy

An 8 year old male horse suffers from tendinopathy in his left fore leg—tenderness on palpation and pain, often when exercising or with movement. The horse exhibits a limp. To treat the animal, autologous MSC at a dose of 1×10⁷ cells are administered via injection to the affected tendon.

Within a week, the horse is no longer exhibiting signs of pain or tenderness in his left fore leg.

### Example 6—Treatment of Tendinopathy

A 4 year old female dog suffers from tendinopathy in her right fore leg—tenderness on palpation and pain, often when exercising or with movement. The dog exhibits a limp. To treat the animal, autologous MSC at a dose of 1×10⁵ cells are administered via injection to the affected tendon.

Within a week, the dog is no longer exhibiting signs of pain or tenderness in her right fore leg.

### Example 7—Treatment of Rheumatoid Arthritis

A 14 year old dog suffers from arthritis. Autologous MSC are administered at a dose of 2.5×10⁷ cells via injection. Within a week, the patient's arthritis symptoms decrease.

### Example 8—Treatment of Lupus

A 3 year old cat suffers from lupus. Allogenic placental MSC at a dose of 2.5×10⁴ cells are administered via injection. Within a week, the patient's lupus symptoms decrease.

### Example 9—Treatment of Tendinopathy

An 11 year old female horse suffers from a ligament tear in his left rear leg—tenderness on palpation and pain, often when exercising or with movement. The horse exhibits a limp. To treat the animal, autologous MSC at a dose of 5×10¹² cells are administered via injection to the affected area.

Within two weeks, the horse is no longer exhibiting signs of pain or tenderness in her left leg.

### Example 10—Treatment of Recurrent Uveitis

A 2 year old female horse suffers from Equine Recurrent Uveitis (ERU). To treat the animal, autologous MSC at a dose of 2.5×10¹² cells are administered via injection to the affected area.

Within a week, the patient's ERU symptoms decrease.

### Example 11—Energy Boost

A 12 year old male horse is lethargic. To treat the animal, allogenic placental MSC at a dose of 5×10¹³ cells are administered via injection.

Within a week, the patient is more energetic.

### Example 12—Treatment of Atopic Dermatitis

A 4 year old dog suffers from atopic dermatitis. Autologous MSC are administered at a dose of 2.5×10⁵ cells via injection. Within a week, the patient's symptoms decrease.

### Example 13—Treatment of Atopic Dermatitis

A 13 year old dog suffers from atopic dermatitis. Allogenic placental MSCs are administered at a dose of 1×10⁷ cells via injection. Within two weeks, the patient's symptoms decrease.

### Example 14—Treatment of Immune Mediated Nephropathy

A 5 year old dog suffers from immune mediated nephropathy. Allogenic MSCs are administered at a dose of 2.5×10⁹ cells via injection. Within three weeks, the patient's symptoms decrease.

### Example 15—Treatment of Chronic Oral Inflammation

A 7 year old cat suffers from chronic oral inflammation. Allogenic MSCs are administered at a dose of 2.5×10⁹ cells via injection. Within two weeks, the patient's symptoms decrease.

### Example 16—Treatment of Chronic Kidney Disease

A 14 year old cat suffers from chronic kidney disease. Autologous MSCs are administered at a dose of 5×10⁸ cells via injection. Within two weeks, the patient's symptoms decrease.

### Example 17—Isolation of IL-10-Producing MSC Population

MSC are harvested as described supra. MSC are then isolated (using MACS) based on their ability to produce IL-10, in order to create a cell product with maximum anti-inflammatory properties.

### Example 18—Characterization of IL-10-Producing MSC Population

MSC are harvested as described supra. MSC lots are stimulated and evaluated for the amount of IL-10 secretion. MSC are classified as negative for IL-10, low expressing IL-10, or high expressing IL-10 lots. This data is then used to correlate the clinical efficacy of MSC lots with their IL-10 production capacity.

### Example 19—Isolation of VEGF-Producing MSC Population

MSC are harvested as described supra. MSC are then isolated (using MACS) based on their ability to produce VEGF to create a cell product with maximum angiogenic property. The product is used for diabetic wound and ischemic diseases.

### Example 20—Characterization of VEGF-Producing MSC Population

MSC are harvested as described supra. MSC are stimulated and evaluated for the amount of VEGF secretion. MSC lots are classified as negative for VEGF, low expressing VEGF, or high expressing VEGF lots. This data is then used to correlate the clinical efficacy of MSC lots with their VEGF production capacity.

### Example 21—Isolation of MMP9-Producing MSC Population

MSC are harvested as described supra. MSC are then isolated (using MACS) based on their ability to produce Matrix Metalloproteinase 9 (MMP9) to create a cell product with maximum anti-fibrotic properties.

### Example 22—Characterization of MMP9-Producing MSC Population

MSC are harvested as described supra. MSCs are stimulated and evaluated for the amount of MMP9 secretion. MSC lots are classified as negative for MMP9, low expressing MMP9, or high expressing MMP9 lots. This data is then used to correlate the clinical efficacy of MSC lots with their MMP9 production capacity.

### Example 23—Activation of IL-10-Producing MSC Population

MSC are harvested as described supra and tested for IL-10 expression. High IL-10 producing MSC lots are then stimulated with inflammatory cytokines with or without C-reactive protein to enhance their anti-inflammatory properties.

### Example 24—Activation of VEGF-Producing MSC Population

MSC are harvested as described supra and tested for VEGF expression. High VEGF producing MSC lots are subjected to low oxygen condition (hypoxia) or high bicarbonate condition to enhance their angiogenic properties.

### Example 25—Activation of MMP9-Producing MSC Population

MSC are harvested as described supra and tested for MMP9 expression. High VEGF-producing MSC lots are treated with various concentrations of ECM components such as different collagen subtypes with or without TGF-beta to enhance their anti-fibrotic properties.

### Example 26—Activation of VEGF-Producing MSC Population

MSC are harvested as described supra and tested for VEGF expression. High VEGF producing MSC lots are subjected to low oxygen condition (hypoxia) or high bicarbonate condition to enhance their angiogenic properties.

### Example 27—Activation of MMP9-Producing MSC Population

MSC are harvested as described supra and tested for VEGF expression. High VEGF producing MSC lots are subjected to various concentration of ECM components such as different collagen subtypes with or without TGF-beta to enhance their anti-fibrotic properties.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for treating an inflammatory disease comprising administration of MSC to a patient in need thereof.

2. The method of claim 1 wherein said patient is a mammal.

3. The method of claim 2 wherein said patient is a human.

4. The method of claim 2 wherein said patient is a cat, dog, or horse.

5. The method of claim 4 wherein said inflammatory disease is chronic inflammation.

6. The method of claim 1, wherein said administration comprises at least one of subcutaneous, intra-articular, intra-lesional, intravenous, intra-peritoneal or intramuscular administration

7. The method of claim 6, wherein said MSC are autologous.

8. The method of claim 6, wherein said MSC are allogenic.

9. The method of claim 7 or 8 wherein said MSC are administered in a dose between 1×10³ cells and 1×10¹² cells.

10. A method for treating arthritis comprising administration of MSC to a mammalian patient in need thereof.

11. The method of claim 10 wherein said patient is a mammal.

12. The method of claim 11 wherein said arthritis comprises ortheoarthritis.

13. The method of claim 10, wherein said administration comprises at least one of subcutaneous, intra-articular, intra-lesional, intravenous, intra-peritoneal or intramuscular administration

14. The method of claim 13, wherein said MSC are autologous.

15. The method of claim 13, wherein said MSC are allogenic.

16. The method of claim 14 or 15 wherein said MSC are administered in a dose between 1×10³ cells and 1×10¹² cells.

17. A method for treating a musculo-skeleton condition comprising administration of placental MSC to a mammalian patient in need thereof.

18. The method of claim 17 wherein said patient is a cat, dog, or horse.

19. The method of claim 18 wherein said immune-related disease is arthritis.

20. The method of claim 17, wherein said administration comprises at least one of subcutaneous, intra-articular, intra-lesional, intra venous, intra-peritoneal or intra muscular administration

21. The method of claim 17, wherein said MSC are autologous.

22. The method of claim 17, wherein said MSC are allogenic.

23. The method of claim 21 or 22 wherein said MSC are administered in a dose between 1×10³ cells and 1×10¹² cells.

24. A method of increasing IL-10 levels in peripheral blood, said method comprising administration of stem cells.

25. The method of claim 24, wherein said stem cells comprise adipose-derived MSC.

26. A method of increasing Interleukin 1 receptor antagonist (IRAP) levels in peripheral blood, said method comprising administration of stem cells.

27. The method of claim 26, wherein said stem cells comprise adipose-derived MSC.

28. A method of reducing lameness in a companion animal, said method comprising administration of stem cells.

29. The method of claim 28, wherein said stem cells comprise adipose-derived MSC.

30. A method of activating an IL-10-producing MSC, comprising stimulating the MSC with inflammatory cytokines.

31. The method of claim 30, further comprising stimulating the MSC with C-reactive protein.

32. A method of activating a VEGF-producing MSC, comprising stimulating the MSC with a hypoxic environment.

33. A method of activating a MMP9-producing MSC, comprising stimulating the MSC with collagen.

34. The method of claim 33, further comprising stimulating the MSC with TGF-beta.
